SEQUENCE LISTING (5 sequences)

SEQ ID NO: 1 - RNA, 16 nt, Artificial Sequence
  Synthetic Construct
uucaagcgauucuccu

SEQ ID NO: 2 - DNA, 36 bp, Artificial Sequence
  Synthetic Construct
aggctgaggcaggagaatcgcttgaaccaaggaggc

SEQ ID NO: 3 - DNA, 84 bp, Artificial Sequence
  Synthetic Construct
ccccccttcaagcgattctcctgtgatccatggtagcgaaggttttctctccacataagggaatacatgatcactgagagctaa

SEQ ID NO: 4 - DNA, 59 bp, Artificial Sequence
  Synthetic Construct
ggggggttagctctcagtgatccatggtagcgaaggttggagagaaaaccttcgctaaa

SEQ ID NO: 5 - DNA, 35 bp, Artificial Sequence
  Synthetic Construct
gcaaaaccttcgctaccatggatcacaacgtctct

(19) United States
(12) Patent Application Publication
(10) Pub. No.: US 2010/0055685 A1
(43) Pub. Date: Mar. 4, 2010

(54) ISOTHERMAL DETECTION METHODS AND USES THEREOF

(75) Inventor: Davis Jame Saul, AUCKLAND (NZ)

Correspondence Address: MORRISON & FOERSTER LLP, 425 MARKET STREET, SAN FRANCISCO, CA, 94105-2482, US

(73) Assignee: ZYGEM CORPORATION LIMITED, HAMILTON (NZ)

(21) Appl. No.: 12/374,996

(22) PCT Filed: Jul. 24, 2007
(86) PCT No.: PCT/NZ2007/000197

§ 371 Date: Oct. 30, 2009

(30) Foreign Application Priority Data
Jul. 24, 2006 (NZ) 548731

(51) Int. Cl. C12Q 1/68

(52) U.S. Cl. 435/6

(57) ABSTRACT

The present invention relates to methods and probes for rapid, single temperature (isothermal) detection of specific nucleic acid sequences. The methods and probes provide an easily automatable system for detecting bioagents including bacteria and viruses, and the detection of specific genetic markers on any nucleic sequence.

### FIELD OF THE INVENTION

This invention relates to the fields of nucleic acid chemistry and molecular genetics. More specifically, it relates to the use of multi-element polynucleotide probes used in combination with nuclease enzymes for detecting specific nucleic acid sequences in biological samples.

### BACKGROUND OF THE INVENTION

Many situations arise where it is desirable to detect low levels of specific nucleic acid sequences within the context of a complex mixture. Examples include, but are not limited to, the detection of medical or environmental pathogens, or the detection of specific gene alleles for identifying genetic abnormalities. In all cases, a method intended for this purpose must be highly specific and highly sensitive. A preferred method should also be robust, relatively simple in application, and inexpensive. With DNA or RNA detection, a detection system may be necessary that is sensitive enough to detect a single molecule (or at best a few molecules) because one target sequence may represent a single infectious agent that has the potential to cause widespread disease.

No simple method currently exists that can detect directly a single nucleic acid molecule of a specific sequence, and so all currently employed methods include a step or steps which amplify the signal. Because DNA has an inherent ability to make copies of itself, it is possible to use an in vitro replication of target sequence that mimics the in vivo process of cellular replication, thereby amplifying the number of target polynucleotides in a complex mixture such that they may be identified with the required sensitivity. The most widespread method used to achieve this goal is the polymerase chain reaction (PCR; as described in detail in U.S. Pat. Nos. 4,683,195, 4,683,202 and 4,800,159). This method provides a geometric amplification of target molecules by using thermal cycling and a thermostable DNA polymerase. High temperature is used to denature (separate) the two complementary DNA strands, and then lower temperatures facilitate priming and strand synthesis by the polymerase. PCR synthesis methods are thus conducted using a reaction that consists of three steps. Detection of the PCR product can be monitored in real-time via degradation of a downstream oligonucleotide mediated by Taq DNA polymerase possessing a 5′-3′ exonuclease activity (Gelfand, 1993). A modification of the PCR reaction scheme that allows for the amplification and detection of RNA targets is the reverse transcription-PCR(RT-PCR) method, which is a combination of the PCR and a reverse transcriptase reaction, as described in Trends in Biotechnology, 10:146-152 (1992).

The basis of the PCR method as commonly applied in the field of molecular diagnostics is that it achieves the desired signal levels by virtue of amplifying the target nucleic acid, followed by detection of these amplified products. In contrast, the Ligase Chain Reaction (LCR) achieves amplification of signal by a geometric increase in a conformation of the probe itself (Barany, 1991). With this method, DNA ligase joins two oligonucleotides in the presence of a target complementary strand and then this ligated form becomes the complementary oligonucleotide for a second pair of primers. One example application of LCR is in the detection of the sexually transmitted disease Chlamydia. This is sold commercially as a kit. (Roche Cobas, Roche Amplicor plate kit). A downside of LCR is that like PCR, it requires thermal-cycling.

An inherent shortcoming in the methods mentioned above is the requirement for the repeated cycling of the reaction between high and low temperatures—for example, the cycling of temperatures to facilitate each round of template denaturation and primer annealing/extension in the case of PCR). The reaction system is therefore conducted using discontinuous phases or cycles because the reaction is restricted by temperature as described above. Thus, the methods require the use of an expensive thermal cycler that can accurately adjust a wide range of temperatures over time. This is specialised equipment that is difficult to miniaturize, and this requirement has limited the application of PCR-based molecular diagnostics to point-of-use testing. Furthermore, the methods require time for adjusting the temperature between the two or three predetermined temperatures. The time lost in adjusting temperature increases in proportion to the cycle number.

In response to this limitation, much effort has been expended to develop single-temperature (isothermal) equivalents of these reactions. In particular, isothermal equivalents of PCR have historically been of particular interest. One approach has been to use a polymerase that simultaneously achieves strand-displacement and strand-synthesis, thereby removing the need for the high-temperature step. Examples of such isothermal nucleic acid amplification methods include the strand displacement amplification (SDA) method as described in JP-B 7-114718, and the various modified SDA methods as described in U.S. Pat. No. 5,824,517, and PCT International patent application publications WO 99/09211, WO 95/25180 and WO 99/49081. In the reactions of these methods, the extension from a primer, and/or the annealing of a primer to a single-stranded extension product or to an original target sequence followed by extension from the primer, takes place in parallel in a reaction mixture incubated at a constant temperature. Where the various methods differ is largely in how they solve the difficulty of primer invasion and annealing. In the original description of SDA, a target nucleic acid sequence (and a complementary strand thereof) in a sample is amplified by displacement of double strands using a DNA polymerase and a restriction endonuclease. The method requires four primers for the amplification, two of which should be designed to contain a recognition site for the restriction endonuclease. The method requires the use of a modified deoxyribonucleotide triphosphate in large quantities as a substrate for DNA synthesis. An example of the modified deoxyribonucleotide triphosphates used in these methods is an (α-S) deoxyribonucleotide triphosphate in which the oxygen atom of the phosphate group at the α-position is replaced by a sulfur atom (S). The incorporation of (α-S) deoxyribonucleotides into the newly synthesised complementary strand of the primer containing the recognition site of the restriction endonuclease creates a hemiphosphorothioate at the cleavage point of the endonuclease. Consequently, the restriction endonuclease nicks only the unmodified strand, facilitating extension of the sequence 5′ of the nick site, and displacement of the strand to the 3′ side of the nick site. However, the expense associated with the use of the modified deoxyribonucleotide triphosphate becomes problematic if the reaction is to be routinely conducted, for example, as a genetic test. Furthermore, the incorporation of the modified nucleotide such as the (α-S) deoxyribonucleotide into the amplified DNA fragment may abolish the cleavability of the amplified DNA fragment with a restriction enzyme, for example, when if is subjected to a restriction enzyme fragment length polymorphism (RFLP) analysis.

The modified SDA method as described in U.S. Pat. No. 5,824,517 is a DNA amplification method that uses a chimeric primer that is composed of RNA and DNA and has as an essential element a structure in which DNA is positioned at least at the 3′-terminus. U.S. Pat. No. 7,056,671 and U.S. Patent Application Publication No. 2003/0073081 relate to another application of chimeric DNA/RNA oligonucleotide primers in an SDA reaction scheme. The modified SDA method as described in PCT International patent application publication WO 99/09211 requires the use of a restriction enzyme that generates a 3′-protruding end. The modified SDA method as described in PCT International patent application publication WO 95/25180 requires the use of at least two pairs of primers. The modified SDA method as described in PCT International patent application publication WO 99/49081 requires the use of at least two pairs of primers and at least one modified deoxyribonucleotide triphosphate. The modified SDA method described in U.S. Patent Application Publication No. 2005/0136417 utilises the action of uracil DNA glycosylase and an apurinic endonuclease to nick one strand of a double stranded DNA moiety, that strand having been synthesised in the presence of dUTP. This effectively creates random priming sites at positions where uracil has been incorporated. In this scheme, adjustment of the ratio of dUTP to dTTP can be used to modulate the frequency of nicking events. These methods can be considered similar to PCR in operation in so far as the sensitive detection of the target nucleic acid is accomplished by target amplification.

The method for synthesizing an oligonucleotide as described in U.S. Pat. No. 5,916,777 comprises synthesizing a DNA oligonucleotide using a primer having a ribonucleotide at the 3′-terminus by completing an extension reaction using the primer, using an endonuclease to introduce a nick between the primer and an extended strand in a primer-extended strand so as to separate the primer and the extended strand, digesting a template and recovering the primer to reuse it. In order to reuse the primer in this method, the primer is isolated from the reaction system and then annealed to the template again.

The LAMP method described in PCT International patent application publication WO 00/28082, which also utilises SDA, employs a set of four primers that recognise six sequences in order form an intermediate with looped ends that is able to be amplified by a strand-displacement polymerase without the need for an intermediate nicking step. In this scheme the original target sequence is amplified in the form of a heterogeneous mixture of concatemeric products of various lengths.

The amplification of circularisable, or “padlock” probes under isothermal conditions in the “Rolling Circle Amplification” (RCA) reaction scheme is another application of SDA (Molecular Diagnosis, 6: 141-150). By using multiple primers with sequences complementary to the circular probe, a branched product is formed via binding of primers to the displaced strands generated by the polymerase. In this “ramified” reaction scheme, the amount of DNA produced increases geometrically. RCA differs from the other SDA schemes discussed above as the product of the reaction is a concatameric array of stretches of DNA having the sequence of or complementary to the circular probe, and not the target DNA.

An example of the commercialization of these SDA strategies includes that of the Eiken Chemical Co. (Japan) who offer diagnostic assays that utilise LAMP technology to detect a variety of bacterial and viral pathogens. Additionally, Becton-Dickson offer a molecular diagnostic platform for the diagnosis of Chlamydia trachomatis and Neisseria gonorrhoeae, based on SDA technology with a restriction endonuclease-mediated cycling strategy.

Another method that employs nicking to facilitate the cycling in an isothermal amplification application is the method described in Proceedings of the National Academy of Sciences, 100: 4504-4509 (2002), U.S. Pat. Nos. 7,112,423 and 6,884,586 and PCT International patent application PCT/US02/22657 published as WO03/008622. In this case, nicking (the cleavage of only one strand of a nucleic acid duplex) is achieved by use of a mutated restriction endonuclease which is able to cut only one strand of the product formed from an initial primer extension step. In one embodiment, subsequent rounds of nicking and extension result in the linear amplification of short oligonucleotides. In another embodiment, (termed exponential amplification reaction/EXPAR), the template used for the initial primer extension step contains a tandem repeat of the primer sequence, such that the products generated from one template strand are able to bind further template strands and act as primers for further extension and nicking reactions, thus generating a geometric increase in the amount of oligonucleotide present. In contrast to the SDA method, this reaction is performed at a temperature that is sufficiently high that the products generated from the nicking reaction dissociate from the template strand without the need for a strand displacement DNA polymerase. However, in the scientific publication (Proceedings of the National Academy of Sciences, 100: 4504-4509 (2002)) a DNA polymerase is used that has strand displacement activity.

Another approach that has been applied to the problem of eliminating thermal cycling from PCR is the use of various DNA binding proteins to enable primer binding and extension without thermal denaturation of the template DNA. Helicase proteins, both in the presence and absence of single-stranded binding proteins, have been applied to the separation of strands of double-stranded DNA to facilitate primer binding, and subsequent extension in the Helicase Dependent Amplification (HDA) method (U.S. Patent Application Publication No.s 2004/0058378 and 2006/0154286). Recombinase proteins have been used to facilitate successive rounds of primer binding to target double-stranded nucleic acids (PLOS Biology, 4:1115-1121 and U.S. Patent Application Publication No. 2007/0054296, 2005/0112631, and 2003/0219792).

HDA technology is the basis of the commercially-available IsoAmp II Universal HDA kits available from New England Biolabs. At present the technology is only applicable to targets in the range of 70-120 nucleotides in length.

Another approach to achieve the isothermal amplification of a target nucleic acid is to exploit the activity of an RNA-polymerase which is able to generate multiple RNA transcripts of a given dsDNA template having the appropriate promoter sequences also present. This is the basis of the self-sustained sequence replication (3SR) method, the nucleic acid sequence based amplification (NASBA) method as described in Japanese Patent No. 2650159, and the transcription-mediated amplification (TMA) method. The Qβ replicase method as described in Japanese Patent No. 2710159 is also conceptually similar, although it exploits the RNA polymerase activity of the Qβ replicase protein. While these methods may be used to produce multiple copies of a specific target sequence, they may also be employed to produce multiple copies of a reporter transcript that is unrelated to the target nucleic acid, as illustrated in the modified method described in Nucleic Acids Research, 29: 54-61 (2001).

The above-mentioned methods enable the amplification of a sufficient amount of target nucleic acid of interest to allow detection. Once sufficient target is available, a number of strategies can used to generate a detectable signal. In that past, this would be most commonly achieved by using radioactive- or immuno-labelling, immunofluorescence labels or by gel electrophoresis. A more elegant development is the use of Fluorescent Resonance Energy Transfer (FRET; Kidwell 1994). FRET makes use of a quantum effect whereby a fluorescent molecule is quenched when in proximity a second molecule—known as a quencher. One early implementation of FRET was with Molecular Beacons (Tyagi and Kramer, 1996; reviewed Broude 2005). Here, stem-loop oligonucleotides are used where the fluorophore and the quencher are on opposite ends of the molecule, but are brought together by base-pairing across the hairpin stem. In the presence of a specific target sequence, the stem is disrupted by preferential base-pairing and the conformational change separates the fluorophore and the quencher thereby increasing fluorescence.

By far the most commonly used application of FRET is the TaqMan system (Livak, 1998) which is an enhancement on the real-time amplification/detection method of Gelfand. In this method, commonly known as real-time PCR, the bound, dual-labelled probe is cleaved by the exonuclease activity of Taq DNA polymerase (or an equivalent) during the extension phase of the PCR. This requirement limits the applicability of the TaqMan technology to non-isothermal amplification systems. Isothermal methods use a polymerase that has displacement activity rather than exonuclease activity (see above). As a consequence, these polymerases will not cleave TaqMan probes and so will not generate detectable signal. However, a number of isothermal nucleic acid detection strategies utilising the principal of FRET have been documented.

Analytical Biochemistry, 333: 246-255 (2004) and U.S. Pat. Nos. 4,876,187 and 5,011,769 describe an application of cycling probe technology for detecting a target nucleic acid by hybridisation with a chimeric DNA/RNA probe labelled with a fluorophore and quencher, in the presence of RNAse H. Binding of the probe to the target generates a RNA/DNA duplex at the chimeric residues, which is a substrate for RNAse H. Hydrolysis of the RNA portion of the probe by RNAse H results in the generation of a fluorescent signal, and allows the probe to dissociate from the target, enabling a second probe to anneal, and trigger another round of signal generation. The result is a linear signal amplification arising from the degradation of the probe in a reaction that is catalysed by the presence of a specific nucleic acid target. A similar strategy has also been employed as a post-PCR genotyping strategy, as described in Clinical Chemistry, 52: 1855-1863 (2006). In this reaction, the presence of a specific target nucleic acid sequence enables the formation of a three-way junction structure, comprising the target nucleic acid; an anchor oligonucleotide containing a phosphorothioate-modified restriction enzyme recognition site; and a reporter oligonucleotide, being partially complementary to the anchor oligonucleotide in the region of the restriction endonuclease recognition site, and possessing a fluorophore and a quencher. Association of the reporter and anchor with the target enables binding of the complementary regions, rendering the restriction enzyme recognition site double stranded, and allowing the reporter oligonucleotide to be cleaved by the appropriate restriction endonuclease. The cleaved reporter oligonucleotide is then able to dissociate from the complex, permitting the binding of a new reporter oligonucleotide.

U.S. Patent Application Publication No. 2004/0101893 employs an apurinic endonuclease to cleave a fluorescent reporter from one end of a fluorescent probe by creating a structure resembling an abasic site from two oligonucleotides that anneal to adjacent regions of the target nucleotide. In this scheme, cleavage of the probe does not result in generation of substantially shorter fragments, and hence is not accompanied by dissociation of the probe from the target as occurs for the other reaction schemes described herein. Another method of FRET-based isothermal signal amplification to detect the presence of specific nucleic acid sequences is that described in Nature Protocols, 1: 554-558 (2006). This method utilises a “sensing” oligonucleotide, which forms a hairpin at both ends. The presence of a target nucleic acid changes the conformation of this oligonucleotide, allowing one end to be cleaved by the restriction enzyme FokI. The resulting product is then able to catalyse the digestion of a portion of a second “fuel” oligonucleotide, which separates a fluorophore and quencher (giving an increase in signal), and allows that oligonucleotide to bind FokI and catalyse the degradation of another “fuel” oligonucleotide (thus propagating the reaction).

Two further methods, relating to strategies for “visible nucleic acid sensing” are documented in Angewandte Chemie International Edition, 45: 2879-2883 (2006) and Organic and Biomolecular Chemistry, 5: 223-225 (2007). In these cases a luminescent or colourimetric reaction is initiated by the presence of a target oligonucleotide, rather than generating a fluorescent signal using a FRET-based system. In the first method, a “Molecular Becon”-type hairpin mRNA oligonucleotide is utilised, having a luciferase or β-galactosidase open reading frame in the 3′ portion, a ribosome binding site in the stem portion, and a loop portion which is complementary to the target nucleic acid. In the presence of the target, the hairpin is opened, freeing the ribosome binding site from the complementary strand, and allowing translation of the luciferase gene, thus generating a luminescent or colourimetric signal. RNAse H is used to degrade part of the hairpin in the presence of the target nucleic acid, resulting in a constitutively free ribosome binding site, and permitting recycling of the target nucleic acid. In the second method, the presence of the target nucleic acid primes a rolling circle amplification (RCA) reaction from a circular probe containing multiple copies of the reverse complement of a DNAzyme having peroxidase activity. Presence of the target thus produces multiple copies of the DNAzyme, which in turn catalyse a colourimetric reaction.

At present, isothermal technologies for sensing of specific nucleic acid sequences via target-induced changes in probe conformation have not been widely applied. The most robust methodologies such as Cycling Probe Technology are limited in sensitivity by virtue of their linear (rather than geometric) amplification characteristics. Hence there remains a need for a detection system able to provide a geometric signal amplification and detection in a single isothermal reaction, without the need for target nucleic acid amplification. It is an object of the present invention to achieve these desiderata, that goes some way to overcoming the disadvantages inherent or present in currently available techniques, or which at least provides a useful choice over existing approaches.

### BRIEF DESCRIPTION OF THE INVENTION

Accordingly, in a first aspect the present invention provides a method for detecting a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridisable to the first nucleic acid molecule, the second nucleic acid molecule comprising at least one copy of the target nucleic acid sequence, or a detection sequence, or both,
c) contacting the sample with more than one copy of the probe, wherein the target binding domain binds the target nucleic acid sequence,
d) contacting the sample with a first nuclease to cleave the nuclease cleavage element or degrade the target binding domain,
e) separating the first and second nucleic acid molecules of the dimeric probe to expose at least one copy of the target nucleic acid sequence on the second nucleic acid molecule, or the detection sequence, or both, wherein this separation allows the target binding domain of the probe to bind at least one copy of the target nucleic acid sequence on the second nucleic acid molecule, and the exposure of additional copies of at least one copy of the target nucleic acid sequence, or the detection sequence, or both,
f) detecting the amount of the target nucleic acid sequence, or the detection sequence, or both.

It should be understood that in so far as the reactions taking place in the method of the present invention is concerned, the method comprises the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridized to the first nucleic acid molecule, the second nucleic acid molecule comprising at least one copy of the target nucleic acid sequence, or a detection sequence, or both,
c) contacting the sample with an excess of the probe so the target binding domain binds the target nucleic acid sequence,
d) contacting the sample with a first nuclease to cleave the nuclease cleavage element or degrade the target binding domain,
e) separating the first and second nucleic acid molecules of the dimeric probe to expose at least one copy of the target nucleic acid sequence on the second nucleic acid molecule, or the detection sequence, or both,
f) allowing the target binding domain of the excess probe to bind at least one copy of the target nucleic acid sequence on the second nucleic acid molecule,
g) repeating steps d) and e) to expose additional copies of the at least one copy of the target nucleic acid sequence, or the detection sequence, or both,
h) repeating steps f) and g) to expose a desired amount of the target nucleic acid sequence, or the detection sequence, or both, and
i) detecting the amount of the target nucleic acid sequence, or the detection sequence, or both.

In various embodiments, the steps a) to h) are carried out sequentially or simultaneously.

In one embodiment, the nuclease cleavage element comprises one strand (the monomeric nucleic acid sequence component) of a restriction endonuclease recognition site, and the first nuclease is a restriction endonuclease.

In one embodiment, the nuclease cleavage element comprises RNA, and the first nuclease is an RNAase, more preferably; RNAse H.

In one embodiment, the separation of the first and second nucleic acid molecules of the dimeric probe is by exonucleolytic degradation of the first nucleic acid molecule by a second nuclease.

In another embodiment, the separation of the first and second nucleic acid molecules of the dimeric probe is by strand displacement by a polymerase having strand displacement activity.

In one embodiment, the first nucleic acid molecule contains a detectable label. Preferably the signal of the detectable label is diminished or rendered undetectable when in sufficiently close proximity to a masking group, and the second nucleic acid molecule contains a masking group capable of diminishing or rendering undetectable the signal of the label when in sufficiently close proximity to the detectable label.

In this embodiment, when the dimeric probe is intact, for example when the first nucleic acid molecule is bound to the second nucleic acid molecule, the detectable label and the masking group are in sufficiently close proximity that the masking group diminishes or renders undetectable the signal of the detectable label. The cleavage of the nuclease cleavage element, or the separation of the first and second nucleic acid molecules, leads to a separation of the detectable label and the masking group sufficient to diminish or prevent the masking of the signal by the masking group.

Preferably, the step of detecting the amount of the target nucleic acid sequence, or the detection sequence, or both is by detecting or measuring the separation of label and masking group by detecting or measuring an increase in the signal of the label as compared to the signal of the intact dimeric probe, wherein an increase in signal is indicative of the presence of said target nucleic acid in the sample.

In another embodiment, the step of detecting the amount of the target nucleic acid sequence, or the detection sequence, or both is by the additional step of contacting the detection sequence with a second probe which hybridises to the detection sequence, the second probe containing a detectable label. Preferably, the second probe additionally contains a masking group that diminishes or renders undetectable the signal of the detectable label when the second probe is not bound to the detection sequence, and wherein the binding of the second probe to the detection sequence leads to a separation of the detectable label and the masking group sufficient to diminish or prevent the masking of the signal by the masking group, wherein an increase in signal of the detectable label is indicative of the presence of said target nucleic acid in the sample. More preferably the second probe is an RMD probe as described herein.

In another aspect the invention provides a method for increasing the number of copies of a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridized to the first nucleic acid molecule, the second nucleic acid molecule comprising at least one copy of the target nucleic acid sequence, or a detection sequence, or both,
c) contacting the sample with an excess of the probe so the target binding domain binds the target nucleic acid sequence,
d) contacting the sample with a first nuclease to cleave the nuclease cleavage element or degrade the target binding domain,
e) separating the first and second nucleic acid molecules of the dimeric probe to expose at least one copy of the target nucleic acid sequence on the second nucleic acid molecule, or the detection sequence, or both,
f) allowing the target binding domain of the excess probe to bind at least one copy of the target nucleic acid sequence on the second nucleic acid molecule,
g) repeating steps d) and e) to expose additional copies of at least one copy of the target nucleic acid sequence, or the detection sequence, or both, and
h) repeating steps f) and g) to expose a desired amount of the at least one copy of the target nucleic acid sequence, or the detection sequence, or both.

The nature of the method allows for the amplification of binding sites for the target binding domain of the dimeric probe, without the contemporaneous synthesis of target nucleic acid.

In a further aspect the present invention provides a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridized to the first nucleic acid molecule, the second nucleic acid molecule comprising at least one copy of the target nucleic acid sequence, or a detection sequence, or both.

In one embodiment, at least one nucleic acid molecule of the dimeric probe is circular.

In another embodiment, the dimeric probe is linear. Preferably, the target binding domain is located at the 5′ terminus, the 3′ terminus, or both termini, of the first strand.

Preferably either or both of the first and second nucleic acid molecules are not susceptible to exonucleolytic activity in the absence of cleavage of the nuclease cleavage element, more preferably the 5′ terminus, the 3′ terminus, or both termini of either or both nucleic acid molecules contains a blocking group capable of blocking exonuclease activity.

In one embodiment, the nuclease cleavage element is one strand (the monomeric nucleic acid sequence component) of a restriction endonuclease recognition site, whereby when bound to target sequence, the target binding domain forms a restriction endonuclease recognition site.

In one embodiment, when said target nucleic acid is DNA, said nuclease cleavage element comprises RNA.

Preferably the detectable label is a fluorophore and said masking group is a quencher capable of quenching the fluorescence of said fluorophore when in sufficiently close proximity.

In a particularly preferred embodiment, the invention provides a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule also carrying a fluorophore, the probe comprising a second nucleic acid molecule hybridized to the first nucleic acid molecule, the second nucleic acid molecule comprising at least one copy of the target nucleic acid sequence and carrying a quencher.

Preferably, said fluorophore is positioned 5′ to the cleavage element of the first nucleic acid molecule.

Preferably, said quencher is positioned 5′ to at least one copy of the target nucleic acid sequence of the second nucleic acid molecule.

More preferably, said quencher is positioned 5′ to the nuclease cleavage element within at least one copy of the target nucleic acid sequence of the second nucleic acid molecule.

Still more preferably, when there is more than one copy of the target nucleic acid sequence of the second nucleic acid molecule, the quencher is positioned 5′ to the nuclease cleavage elements of the all of the more than one copy of the target nucleic acid sequence.

In another aspect, the invention provides a method for detecting a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule carrying a quencher and comprising at least one copy of the target nucleic acid sequence, the probe comprising a second nucleic acid molecule hybridized to the first nucleic acid molecule, the second nucleic acid molecule carrying a fluorophore,
c) contacting the sample with an excess of the probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a fluorescent signal and exposing the at least one copy of the target nucleic acid sequence on the first nucleic acid molecule, wherein this exposing allows the target binding domain of the probe to bind the exposed at least one copy of the target nucleic acid sequence on the first nucleic acid molecule, and the amplification of the fluorescent signal and exposure of additional copies of the at least one copy of the target nucleic acid sequence, and
f) detecting or measuring the fluorescent signal,
wherein an increase in signal is indicative of the presence of the target nucleic acid in the sample.

It should be understood that in so far as the reactions taking place in the method of the present invention is concerned, the method comprises the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule carrying a quencher and comprising at least one copy of the target nucleic acid sequence, the probe comprising a second nucleic acid molecule hybridized to the first nucleic acid molecule, the second nucleic acid molecule carrying a fluorophore,
c) contacting the sample with an excess of the probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a fluorescent signal and exposing one copy of the target nucleic acid sequence on the first nucleic acid molecule,
f) allowing the target binding domain of the excess probe to bind at least one copy of the target nucleic acid sequence on the first nucleic acid molecule,
g) repeating steps d) and e) to amplify the fluorescent signal and expose additional copies of the target nucleic acid sequence, and
h) repeating steps f) and g) to amplify the fluorescent signal to a desired level,
i) detecting or measuring the fluorescent signal,
wherein an increase in signal is indicative of the presence of the target nucleic acid in the sample.

In various embodiments, the steps a) to h) are carried out sequentially or simultaneously.

In still a further aspect, the invention provides a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule carrying a quencher and comprising at least one copy of the target nucleic acid sequence, the probe comprising a second nucleic acid molecule hybridized to the first nucleic acid molecule, the second nucleic acid molecule carrying a fluorophore.

In another aspect, the invention provides a method for detecting a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second, circular nucleic acid molecule hybridisable to the first nucleic acid molecule, the second nucleic acid molecule comprising at least one copy of a sequence that is the reverse complement of the target nucleic acid sequence and at least one copy of a sequence that is the reverse complement of a detection sequence,
c) contacting the sample with more than one copy of the dimeric probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a reverse complement of the second nucleic acid molecule, the reverse complement containing at least one copy of the target nucleic acid sequence and at least one copy of the detection sequence, wherein the generation allows the target binding domain of the probe to bind the exposed at least one copy of the target nucleic acid sequence and the exposure of additional copies of the at least one copy of the target nucleic acid sequence, or the detection sequence, or both, and
f) contacting the sample with a second probe that binds the detection sequence, the second probe carrying a detectable label,
g) detecting or measuring the signal of the detectable label,
wherein an increase in signal is indicative of the presence of the target nucleic acid in the sample.

It should be understood that in so far as the reactions taking place in the method of the present invention is concerned, the method comprises the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second, circular nucleic acid molecule hybridized to the first nucleic acid molecule, the second nucleic acid molecule comprising at least one copy of a sequence that is the reverse complement of the target nucleic acid sequence and at least one copy of a sequence that is the reverse complement of a detection sequence,
c) contacting the sample with an excess of the dimeric probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a reverse complement of the second nucleic acid molecule containing at least one copy of the target nucleic acid sequence and at least one copy of the detection sequence,
f) allowing the target binding domain of the excess probe to bind the exposed at least one copy of the target nucleic acid sequence on the reverse complement of the second nucleic acid molecule,
g) repeating steps d), e), and f) to expose additional copies of the at least one copy of the target nucleic acid sequence, or the detection sequence, or both, and
h) contacting the sample with a second probe that binds the detection sequence, the second probe carrying a detectable label,
i) detecting or measuring the signal of the detectable label,
wherein an increase in signal is indicative of the presence of the target nucleic acid in the sample.

In various embodiments, the steps a) to h) are carried out sequentially or simultaneously.

Preferably, the second probe is a single stranded RNA probe comprising a fluorophore, a quencher and a detection sequence binding domain, more preferably the second probe is an RMD probe as described herein.

In still a further aspect, the invention provides a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second, circular nucleic acid molecule hybridized to the first nucleic acid molecule, the second nucleic acid molecule comprising at least one copy of a sequence that is the reverse complement of the target nucleic acid sequence and at least one copy of a sequence that is the reverse complement of a detection sequence.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the probe comprising a target binding domain and at least one copy of the target nucleic acid sequence.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the first nucleic acid molecule comprising a target binding domain and the second nucleic molecule comprising at least one copy of the target nucleic acid sequence.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the probe comprising a target binding domain and at least one copy of the target nucleic acid sequence, wherein the at least one copy of the target nucleic acid sequence is available for binding by the target binding domain of another copy of the probe when the first and second nucleic acid molecules of the dimeric probe are separated.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the probe comprising a target binding domain and at least one copy of the target nucleic acid sequence, wherein the at least one copy of the target nucleic acid sequence is masked when the dimeric probe is intact and is available for binding by the target binding domain of another copy of the probe when the first and second nucleic acid molecules of the dimeric probe are separated.

In a further aspect, the invention provides a method for detecting a target DNA in a sample, the method comprising the steps

a) providing a sample containing a target DNA sequence,
b) providing a single stranded RNA probe carrying a detectable label and a masking group, the probe comprising a target binding domain,
c) contacting the sample with the probe so the target binding domain binds the target DNA sequence,
d) contacting the sample with a nuclease to degrade bound probe and separate the detectable label from the masking group, thereby generating a signal,
e) detecting or measuring the signal,
wherein an increase in signal as compared to the signal of the intact probe is indicative of the presence of the target nucleic acid in the sample.

Preferably, the detectable label is a fluorophore and the masking group is a quencher.

Preferably, the nuclease is ribonuclease H(RNAse H) or an agent having RNAse H activity.

In a further aspect, the invention provides a single stranded RNA probe comprising a fluorophore, a quencher and a target binding domain.

In another aspect, the present invention provides a composition containing a probe of the invention, together with one or more additives, buffers, excipients, or stabilisers.

Preferably, the composition additionally contains one or more of the group comprising:

a nuclease;

an exonuclease;

a polymerase having strand displacement activity;

a compound, co-factor or co-enzyme to activate or augment the activity of the nuclease;

a compound, co-factor or co-enzyme to activate or augment the activity of the exonuclease;

a substrate, compound, co-factor or co-enzyme to activate or augment the activity of the polymerase.

In another aspect, the present invention provides a kit for detecting target nucleic acid in a sample, said kit comprising a quantity of dimeric probe of the invention, a quantity of a nuclease, and a quantity of a strand-separating activity, together with instructions for contacting the probe, the nuclease, and the strand-separating activity with the sample.

In one embodiment, the kit additionally comprises a detection probe, preferably the detection probe is a single stranded RNA probe comprising a fluorophore, a quencher and a detection sequence binding domain.

### BRIEF DESCRIPTION OF THE DRAWINGS

Reference can be made to the accompanying drawings in which:

FIG. 1 is a diagram showing the major elements of a Nuclease Chain Reaction (NCR) probe (Panel A). Methods using this probe rely on a restriction endonuclease to catalyse the reaction. The stages involved in a single iteration of the chain reaction are shown in Panels B, C and D.

FIG. 2 is a diagram showing the major elements of an RNAse-mediated Nuclease Chain Reaction (RNCR) probe (Panel A). Methods using this probe rely on an RNA region in one of the strands and use a ribonuclease H enzyme to catalyse the reaction. The stages involved in single iteration of the reaction are shown in Panels B, C and D.

FIG. 3 is a diagram showing the major elements of a Polymerase-Nuclease Chain Reaction (PNCR) probe (Panel A). Methods using this probe rely on a restriction endonuclease to catalyse the reaction and a DNA polymerase with displacement activity to reveal the intrinsic target sites. The stages involved in single iteration of the reaction are shown in Panels B, C and D.

FIG. 4 is a diagram showing the major elements of an RNAse-Mediated Detection (RMD) probe and shows the stages in a single iteration of the reaction.

FIG. 5 shows the fluorescence plot of an RMD reaction over time in the presence of varying quantities of target sequence. The table gives an indication of the sensitivity of the method in the detection of target sequences.

FIG. 6 is a diagram showing the probes and the reaction stages in a method that combines RNCR and RMD.

FIG. 7 is a diagram showing the probe and the stages in a reaction that combines PNCR and RMD in a rolling-circle replication system (RC-PNCR).

FIG. 8 is a diagram showing a typical design for a RC-PNCR probe.

Although the invention is broadly as described above, it will be appreciated by those persons skilled in the art that the invention is not limited thereto but also includes embodiments of which the following description gives examples.

### DETAILED DESCRIPTION OF THE INVENTION

As noted above, in one aspect the present invention is directed to an isothermal detection method to detect target nucleic acid, wherein the method is not reliant on and does not involve target amplification. Rather, the method relies on the target nucleic acid-dependent amplification of signal from a detectable label bound to a nucleic acid probe. Here, signal amplification occurs as a result of the presence of target nucleic acid, but without the amplification of target nucleic acid.

At least part of the nucleic acid probe is able to hybridise with the target nucleic acid. This is referred to herein as the target binding region or target binding domain. Hybridisation of probe and target nucleic acid sequence forms a nuclease cleavage element capable of being cleaved by a nuclease. Cleavage of the nuclease cleavage element ultimately leads to the separation of detectable label from a masking group capable of diminishing or rendering undetectable the signal from the detectable label.

In some embodiments, cleavage of the cleavage element also ultimately reveals at least one further target nucleic acid sequence present within the probe, itself able to hybridise with further probe molecules thereby leading to the formation of further probe:target nucleic acid sequence hybrids. Each of these further hybrids contains a nuclease cleavage element capable of being cleaved by the nuclease. Again, cleavage leads to the separation of further label from masking group, signal emission, exposure of further target nucleic acid sequence present within the further probe molecules, and so on such that a geometric amplification of signal is achieved.

In other embodiments, cleavage of the cleavage element reveals a detection sequence, which is able to be detected, for example by binding to a detection probe comprising a detectable label.

Fundamentally then, the target nucleic acid can be thought of as the catalyst for the separation of label and masking group and the consequent emission of signal.

In one embodiment, the dimeric probes of the invention contain one or more copies of the target nucleic acid sequence or a sequence able to hybridise to the target-binding region of the probe, also referred to below as intrinsic targets. When the probe is intact or in the absence of target nucleic acid, these intrinsic targets are hidden or masked from the nuclease enzyme(s) carrying out the reaction by the complementary oligonucleotide or polynucleotide, conveniently referred to herein as the first nucleic acid molecule. In one embodiment a restriction endonuclease is used in conjunction with an exonuclease. Mismatches are included in the design to prevent spontaneous cleavage of the probe by an endonuclease. This preferred embodiment is referred to herein as the Nuclease Chain Reaction (NCR), and is described in more detail herein in Example 1. In a second preferred embodiment, a ribonuclease H enzyme and exonuclease are used to achieve a similar effect. This embodiment is referred to herein as the Ribonuclease Chain Reaction (RNCR), and is described in more detail in Example 2. In a third preferred embodiment, a displacement DNA polymerase is used to denature the dimeric probe instead of an exonuclease. This embodiment is referred to herein as the Polymerase-Nuclease Chain Reaction (PNCR), and is described in more detail in Example 3.

Once triggered by the presence of a target nucleic acid to be detected (which can be thought of as an “extrinsic” target sequence to distinguish it from the intrinsic copies present in the probe), the inaccessible intrinsic targets are exposed by separating the strands of the dimeric probe, for example, by hydrolysis of one strand of the dimeric probe or by strand displacement. By designing the dimeric probe to contain at least two copies of intrinsic target, a geometric amplification of signal (doubling or tripling) can be achieved at each iteration of the reaction.

In other embodiments, the dimeric probes of the invention contain one or more copies of a detection sequence, the detection sequence being either able to bind to a detection probe, or to encode a sequence able to bind to a detection probe.

Depending on the design of the dimeric probe, the trigger to the chain reaction can include: (i) the formation of a target:probe hybrid between the target-binding domain of the probe and the target DNA, thereby creating a specific endonuclease recognition site, and the subsequent cleavage of the site, (ii) the formation of DNA/RNA target:probe hybrid between an RNA region on the probe and the target DNA, and degradation of the target:probe hybrid by an agent having RNA:DNA hybrid-degrading activity (for example, an agent having ribonuclease H(RNAse H) activity).

In various embodiments, the separation of label and masking group may occur directly as a result of the cleavage event by the nuclease activity, or indirectly, for example as a result of denaturation of the dimeric probe enabled by the cleavage event. For example, such indirect separation may include exonucleolytic degradation of part of the probe by an exonuclease. Whether the separation is direct or indirect will largely be a function of the relative positions of the cleavage element, the masking group and the label within the probe.

In other embodiments, the label and masking group may each be present on a separate probe (for example, a “detection probe” as described herein) able to bind to a detection sequence present within or produced by synthesis encoded by the dimeric probe of the invention, wherein the binding of the detection probe to the detection sequence leads to the separation of the label and the masking group, for example, by RNAase H-mediated cleavage of the detection probe.

Target DNA should be rendered single-stranded prior to the detection, and should be protected from the action of any exonuclease used in the reaction. Endonuclease activity at other sites on the target chromosome is not detrimental to the method. Exonuclease activity can be minimised by complementary PNA blockers flanking the target. The use of PNAs in this manner has a double function in that PNAs are known to cause strand invasion of duplex DNA thereby creating and stabilizing single-stranded regions of DNA (Peffer et al, 1993).

Alternatively, a restriction endonuclease with a nicking activity could be used to reduce the impact of exonuclease on the target DNA. These enzymes cut only one strand of DNA and so by using an exonuclease with minimal nick activity, degradation of the target can be reduced. Placement of the site close to one end of the hybridised region can ensure that once nicked, the number of nucleotides involved in base pairings is reduced to such an extent that the probe is freed from the target thereby generating an end suitable for exonuclease activity. Typical nicking endonucleases are N.BstNB I, N.Alw I, N.BbvC IA and N.BbvC IB.

With linear probes of the present invention, blockers are used in some embodiments to prevent exonuclease activity on the un-triggered probe. These can be any modified form of DNA including amino linkage, thiol linkage, 3′-3′ linkage, 5′-5′ linkage, nucleoside analogues, spacers or 5′ or 3′ terminal modifications including dephosphorylation. In addition, the termini may be blocked with short complementary strands of modified DNA or be blocked by binding proteins.

Accordingly, in a first aspect the present invention provides a method for detecting a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridisable to the first nucleic acid molecule, the second nucleic acid molecule comprising at least one copy of the target nucleic acid sequence, or a detection sequence, or both,
c) contacting the sample with more than one copy of the probe, wherein the target binding domain binds the target nucleic acid sequence,
d) contacting the sample with a first nuclease to cleave the nuclease cleavage element or degrade the target binding domain,
e) separating the first and second nucleic acid molecules of the dimeric probe to expose the at least one copy of the target nucleic acid sequence on the second nucleic acid molecule, or the detection sequence, or both, wherein this separation allows the target binding domain of the probe to bind the exposed at least one copy of the target nucleic acid sequence on the second nucleic acid molecule, and the exposure of additional copies of the at least one copy of the target nucleic acid sequence, or the detection sequence, or both,
f) detecting the amount of the target nucleic acid sequence, or the detection sequence, or both.

It should be understood that in so far as a description of the reactions taking place in the method of the present invention is concerned, the method comprises the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridized to the first nucleic acid molecule, the second nucleic acid molecule comprising at least one copy of the target nucleic acid sequence, or a detection sequence, or both,
c) contacting the sample with an excess of the probe so the target binding domain binds the target nucleic acid sequence,
d) contacting the sample with a first nuclease to cleave the nuclease cleavage element or degrade the target binding domain,
e) separating the first and second nucleic acid molecules of the dimeric probe to expose the at least one copy of the target nucleic acid sequence on the second nucleic acid molecule, or the detection sequence, or both,
f) allowing the target binding domain of the excess probe to bind the exposed at least one copy of the target nucleic acid sequence on the second nucleic acid molecule,
g) repeating steps d) and e) to expose additional copies of the at least one copy of the target nucleic acid sequence, or the detection sequence, or both,
h) repeating steps f) and g) to expose a desired amount of the target nucleic acid sequence, or the detection sequence, or both, and
i) detecting the amount of the target nucleic acid sequence, or the detection sequence, or both.

In various embodiments, the steps a) to h) are carried out sequentially or simultaneously.

In one embodiment, the first nucleic acid molecule contains a detectable label. Preferably the signal of the detectable label is diminished or rendered undetectable when in sufficiently close proximity to a masking group, and the second nucleic acid molecule contains a masking group capable of diminishing or rendering undetectable the signal of the label when in sufficiently close proximity to the detectable label.

In this embodiment, when the dimeric probe is intact, the detectable label and the masking group are in sufficiently close proximity that the masking group diminishes or renders undetectable the signal of the detectable label. The cleavage of the nuclease cleavage element, or the separation of the first and second nucleic acid molecules, leads to a separation of the detectable label and the masking group sufficient to diminish or prevent the masking of the signal by the masking group.

Preferably, the step of detecting the amount of the target nucleic acid sequence, or the detection sequence, or both is by detecting or measuring the separation of label and masking group by detecting or measuring an increase in the signal of the label as compared to the signal of the intact dimeric probe, wherein an increase in signal is indicative of the presence of said target nucleic acid in the sample.

In another embodiment, the step of detecting the amount of the target nucleic acid sequence, or the detection sequence, or both is by the additional step of contacting the detection sequence with a second probe which hybridises to the detection sequence, the second probe containing a detectable label. The second probe is also referred to herein as a “detection” probe.

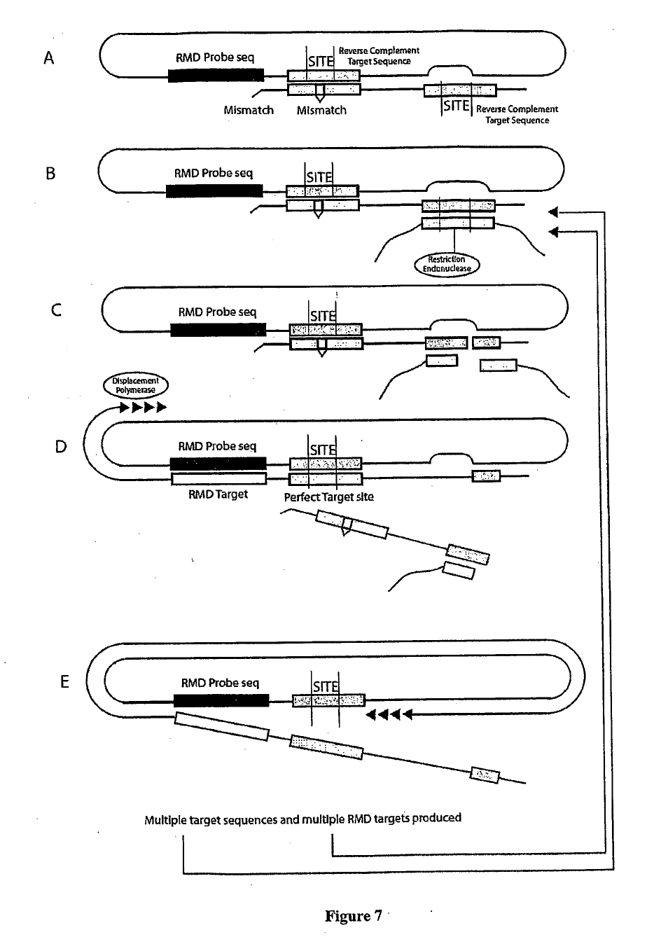

In another embodiment, the detection sequence present in the dimeric probe of the invention is the reverse complement of a sequence able to bind to a detection probe, wherein synthesis of nucleic acid using such a detection sequence as a template will produce a nucleic acid able to bind to a detection probe. In this embodiment, the step of detecting the amount of the target nucleic acid sequence, or the detection sequence, or both is by the additional steps of synthesising the reverse complement of the detection sequence, and contacting the reverse complement of the detection sequence with a second probe which hybridises to the reverse complement of the detection sequence. See, for example, the method described in Example 6 and shown in FIG. 7 herein.

Preferably, the second probe additionally contains a masking group that diminishes or renders undetectable the signal of the detectable label when the second probe is not bound to the detection sequence, and wherein the binding of the second probe to the detection sequence leads to a separation of the detectable label and the masking group sufficient to diminish or prevent the masking of the signal by the masking group, wherein an increase in signal of the detectable label is indicative of the presence of said target nucleic acid in the sample. More preferably the second probe is a single stranded RNA probe (RMD probe) as described herein.

Preferably, the method comprises the additional steps of

h-i) contacting the sample with a second probe that binds the detection sequence, the second probe carrying a detectable label,
i-ii) detecting or measuring the signal of the detectable label,
wherein an increase in signal is indicative of the presence of the target nucleic acid in the sample.

Preferably, the second probe is a single stranded RNA probe comprising a fluorophore, a quencher and a detection sequence binding domain, more preferably the second probe is an RMD probe as described herein.

In one embodiment, the nuclease cleavage element comprises one strand (the monomeric nucleic acid sequence component) of a restriction endonuclease recognition site, and the first nuclease is a restriction endonuclease.

In one embodiment, the nuclease cleavage element comprises RNA, and the first nuclease is an RNAase, more preferably, RNAse H.

In one embodiment, the separation of the first and second nucleic acid molecules of the dimeric probe is by exonucleolytic degradation of the first nucleic acid molecule by a second nuclease.

In another embodiment, the separation of the first and second nucleic acid molecules of the dimeric probe is by strand displacement by a polymerase having strand displacement activity.

It will be apparent to those skilled in the art that in various embodiments, the steps a) to h) are performed in any order, sequentially or simultaneously. The chain reaction and signal amplification is triggered only when the reaction components—the target nucleic acid, the probe, the nuclease, and the strand-separating activity—are all present. In one embodiment, the target nucleic acid may be contacted with a composition containing the dimeric probe, the nuclease, and the strand-separating activity. Advantageously, this minimises the opportunities for introducing contamination when the method is performed in a closed system.

It will be appreciated that the methods of the invention can be performed qualitatively or quantitatively. For example, the methods can give a binary (yes/no) indication of whether the one or more species of target nucleic acid is present in the sample. In another example, the indication may be semi-quantitative, for example, by giving three levels of signal—high, low, and no signal. Depending on the label, these levels could for example be shades of the same colour, wherein a darker shade indicates a high level of target nucleic acid, a medium shade indicates a low level of target nucleic acid, and a light shade or no colour indicates no target nucleic acid present. The methods also provide for the quantitative analysis of target nucleic acid, for example by measurement, including real-time measurement, of the production of signal. An example of such quantitative measurement of target nucleic acid is presented herein in the Examples.

It will be apparent that the dimeric probe, and when used the second, detection probe, is preferably present in molar excess of the target nucleic acid sequence to be detected. In most embodiments it will be preferable to have the probe present in non-limiting molar excess so that the concentration or amount of the probe(s) is/are not rate-limiting. However, in some embodiments it may be desired that the amount or concentration of one or both probes is rate limiting, for example in situations where a qualitative result is desired. Appropriate methods to calculate a suitable amount of probe(s) given the amount or concentration of target nucleic acid or other reaction conditions are well known to those skilled in the art.

The terms “nucleic acid”, “nucleic acid sequence”, “polynucleotide(s),”, “polynucleotide sequence” and equivalents thereof as used herein mean a single or double-stranded deoxyribonucleotide or ribonucleotide polymer of any length, and include as non-limiting examples, coding and non-coding sequences of a gene, sense and antisense sequences, exons, introns, genomic DNA, cDNA, pre-mRNA, mRNA, rRNA, siRNA, miRNA, tRNA, ribozymes, recombinant polynucleotides, isolated and purified naturally occurring DNA or RNA sequences, synthetic RNA and DNA sequences, nucleic acid probes, primers, fragments, genetic constructs, vectors and modified polynucleotides. There is no intended distinction in length between the terms “nucleic acid” and “polynucleotide”, and these terms will be used interchangeably.

The terms “target region”, “target sequence”, “target nucleic acid”, “target nucleic acid sequence”, “target polynucleotide”, and “target polynucleotide sequence” and grammatical equivalents thereof refer to a region of a nucleic acid which is to be detected. The term “target nucleic acid sequence” as used herein therefore includes the target nucleic acid to be detected, for example that present in a sample, and the copies of the target nucleic acid sequence present within the probes of the invention. For example, that sequence present in the second nucleic acid molecule of examples of the dimeric probes of the present invention that, on hybridisation with the target binding domain of the first nucleic acid molecule of probes of the present invention, forms a nuclease cleavage element is herein referred to as a target nucleic acid sequence.

Preferably, the target nucleic acid will be single-stranded, thereby facilitating the formation of a target:probe hybrid. Methods to render the target nucleic acid single-stranded are well-known in the art, and will most commonly involve heat denaturation of double-stranded nucleic acids. Chemical agents that prevent or diminish the formation of base-pairing are also well-known in the art for use in rendering nucleic acids single-stranded. It will be apparent to the skilled artisan that such agents must be used cautiously in the methods of the present invention, as these methods are reliant on the formation of, for example, target:probe hybrids via hybridisation.

It will also be appreciated that some nucleic acids exist that possess “strand invasion” properties, whether such strand invasion results in the displacement of the complementary strand of the target nucleic acid and the formation of a target:probe duplex, or the formation of a target:probe triplex, without the target sequence first being single-stranded. Peptide nucleic acids (PNAs) and derivatives thereof may be capable of strand invasion, whereby probes of the present invention containing target nucleic acid binding regions comprising PNAs can be used to detect target nucleic acid that has not been rendered fully single-stranded. The use of target-binding regions comprising PNAs is particularly contemplated in circular probes of the present invention, where, prior to the formation of the target:probe hybrid, the target-binding region of the probe may be substantially double-stranded.

The term “probe” refers to a polynucleotide used in a hybridisation-based assay to detect a target polynucleotide sequence that is complementary to at least part of the probe. The probe will comprise a target binding domain that hybridises to a region of the target nucleic acid sequence. In various embodiments of the present invention, probes are labeled with, i.e., bound to, a detectable label to enable detection. The probe may consist of a “fragment” of a polynucleotide as defined herein.

“Corresponding” means identical to or capable of hybridising to the reverse complement of the designated nucleic acid.

The term “hybridisation” and grammatical equivalents refers the formation of a multimeric structure, usually a duplex structure, by the binding of two or more single-stranded nucleic acids due to complementary base pairing. Hybridisation can occur between fully complementary nucleic acid strands or between nucleic acid strands that contain minor regions of mismatch. Two single-stranded nucleic acids that are complementary except for minor regions of mismatch are referred to as substantially complementary. Stable duplexes of substantially complementary sequences can be achieved under less stringent hybridization conditions. Those skilled in the art of nucleic acid technology can determine duplex stability empirically considering a number of variables including, for example, the length and base pair concentration of the polynucleotides, ionic strength, and incidence of mismatched base pairs. Conditions for hybridisation can be modified as appropriate, for example to allow only those single-stranded regions with sufficiently high degrees of complementarity to hybridise. Stringent conditions for the hybridisation of highly complementary nucleic acids only are described herein.

As used herein, “duplex-forming region” refers to nucleic acid sequence present in a polynucleotide that is sufficiently complementary to nucleic acid sequence present in another polynucleotide to allow hybridisation of the polynucleotides, and particularly contemplates the one or more regions present in the nucleic acid molecules comprising the dimeric probes of the invention that form a double-stranded region of the intact dimeric probe.

As used herein, “target-binding domain” and its equivalent “target binding domain” refers to nucleic acid sequence present in a nucleic acid molecule that is sufficiently complementary to nucleic acid sequence present in the target nucleic acid to allow the hybridisation of the target-binding region and the target nucleic acid, and so to form a target:probe hybrid.

As used herein, “nuclease cleavage element” refers to nucleic acid sequence present in a probe nucleic acid molecule that forms a region subject to cleavage by a nuclease when hybridised with the target nucleic acid sequence or a sequence corresponding to the target nucleic acid. Preferably, the one or more cleavage elements present in a probe are not susceptible to cleavage so long as the probe is not bound to target nucleic acid. More preferably, any cleavage elements present in the target-binding region of the probe are not susceptible to cleavage so long as the probe is not bound to target nucleic acid, or while the first and second nucleic acid molecules of the probe are hybridised and the probe is intact.

As used herein, nucleases include molecules, compounds, or enzymes, preferably enzymes that are capable of selectively cleaving nucleic acid. Preferably, the nuclease will selectively cleave particular nucleic acid sequences with high specificity. Preferred nucleases will cleave both strands of double-stranded nucleic acids. Endonucleases are examples of preferred nucleases. Many endonucleases, including restriction endonucleases, exist and are well characterised and well known in the art. Any site-specific endonuclease can be used in the methods of the invention, and can be selected in accordance with the design of the target-binding domain of the probe, itself largely determined by the sequence of the target nucleic acid. However, a preferred endonuclease would have a reduced recognition site frequency to minimise fragmentation of the target nucleic acid, for example the chromosome on which the target nucleic acid sequence lies. The choice of nuclease will be determined by availability of appropriate sequences within the potential target regions of the nucleic acid to be detected. For example, if a target nucleic acid sequence contains a recognition site for a particular restriction endonuclease, the target-binding domain of the probe can be designed to incorporate the restriction site, so that that restriction endonuclease can be used to cleave any target:probe hybrids that form.

In the present invention, the first and second nucleic acid molecules of the dimeric probe are separated, preferably by a strand-separating activity. Such activities include molecules, compounds or enzymes, preferably enzymes, that are capable of dissociating the first and second nucleic acid molecules of the dimeric probe. In one embodiment, this dissociation involves the degradation of one, preferably the first, nucleic acid molecule, for example by hydrolysis wherein a preferred agent is an exonuclease. Both 5′-3′ exonucleases and 3′-5′ exonucleases may be used, and can be selected as appropriate given the design of the dimeric probe and the position of the elements within the probe. For example, for a linear probe with a target-binding domain comprising a single-stranded extension at the 5′ end of the first nucleic acid molecule, a 5′-3′ exonuclease is appropriate. Alternatively, 3′-5′ exonucleases may be used when target-binding region comprising a single-stranded extension is placed on the 3′ end of the polynucleotide. Preferred exonucleases have single-stranded and double-stranded exonuclease activity, minimal nick activity and no endonuclease activity. Well-known exonucleases with suitable activity are Lambda exonuclease and T7 exonuclease.

In another embodiment, the separation of the first and second nucleic acid molecules is achieved by dissociation of the double-stranded region(s) of the dimeric probe, wherein a preferred agent having strand-separating activity is a nucleic acid polymerase, preferably a DNA polymerase, having strand-displacement activity. Such polymerases are well-known in the art and are discussed herein.

The methods for detecting target nucleic acids of the present invention are reliant on detecting or measuring the signal from a label, preferably the light emission of a probe labeled with a light-emitting label.

The term “label”, as used herein, refers to any atom, molecule, compound or moiety which can be attached to a nucleic acid, and which can be used either to provide a detectable signal or to interact with a second label to modify the detectable signal provided by the second label. Preferred labels are light-emitting compounds which generate a detectable signal by fluorescence, chemiluminescence, or bioluminescence. Still more preferred labels are light-emitting compounds the signal of which is diminished or rendered undetectable when in sufficiently close proximity to a masking group, for example, a quenching chromophore.

The methods of the invention are applicable to the detection of probes labeled with a single label, although multiple labels may be employed. Detection of the cleaved probe occurs when the label, for example a fluorophore, is sufficiently removed from the masking group, for example a quencher, by the cleavage event, or the probe-denaturing process the cleavage event allows. This diminishes the interaction of the masking group and the label and so allows emission of the signal.

As used herein, the term “masking group” means any atom, molecule, compound or moiety that can interact with the label to decrease the signal emission of the label. The separation of label and masking group resulting from the cleavage event or the probe-denaturing process the cleavage event allows in turn results in a detectable increase in the signal emission of the attached label. Depending on the label, signal emission may include light emission, particle emission, the appearance or disappearance of a coloured compound, and the like. Preferred light-emitting labels and masking groups that can interact to modify the light emission of the label are described below.

The term “chromophore” refers to a non-radioactive compound that absorbs energy in the form of light. Some chromophores can be excited to emit light either by a chemical reaction, producing chemiluminescence, or by the absorption of light, producing fluorescence.

The term “fluorophore” refers to a compound which is capable of fluorescing, i.e. absorbing light at one frequency and emitting light at another, generally lower, frequency.

The term “bioluminescence” refers to a form of chemiluminescence in which the light-emitting compound is one that is found in living organisms: Examples of bioluminescent compounds include bacterial luciferase and firefly luciferase.

The term “quenching” refers to a decrease in fluorescence of a first compound caused by a second compound, regardless of the mechanism. Quenching typically requires that the compounds be in close proximity. As used herein, either the compound or the fluorescence of the compound is said to be quenched, and it is understood that both usages refer to the same phenomenon.

Mechanisms by which the light emission of a compound can be quenched by a second compound are described in Morrison, 1992, in Nonisotopic DNA Probe Techniques (Kricka ed., Academic Press, Inc. San Diego, Calif.), Chapter 13. One well known mechanism is fluorescence energy transfer (FET), also referred to in the literature as fluorescence resonance energy transfer, nonradiative energy transfer, long-range energy transfer, dipole-coupled energy transfer, and Forster energy transfer. The primary requirement for FET is that the emission spectrum of one of the compounds, the energy donor, must overlap with the absorption spectrum of the other compound, the energy acceptor. Styer and Haugland, 1967, Proc. Natl. Acad. Sci. U.S.A. 98:719, incorporated herein by reference, show that the energy transfer efficiency of some common emitter-quencher pairs can approach 100% when the separation distances are less than 10 angstroms. The energy transfer rate decreases proportionally to the sixth power of the distance between the energy donor and energy acceptor molecules. Consequently, small increases in the separation distance greatly diminish the energy transfer rate, resulting in an increased fluorescence of the energy donor and, if the quencher chromophore is also a fluorophore, a decreased fluorescence of the energy acceptor.

In the methods of the present invention, the signal emission of label, preferably a fluorescent label, bound to the probe is detected. Many fluorophores and chromophores described in the art are suitable for use in the methods of the present invention. Suitable fluorophore and quenching chromophore pairs are chosen such that the emission spectrum of the fluorophore overlaps with the absorption spectrum of the chromophore. Ideally, the fluorophore should have a high Stokes shift (a large difference between the wavelength for maximum absorption and the wavelength for maximum emission) to minimize interference by scattered excitation light.

Suitable labels which are well known in the art include, but are not limited to, fluoroscein and derivatives such as FAM, HEX, TET, and JOE; rhodamine and derivatives such as Texas Red, ROX, and TAMRA; Lucifer Yellow, and coumarin derivatives such as 7-Me₂N-coumarin-4-acetate, 7-OH-4-CH.₃-coumarin-3-acetate, and 7-NH₂-4-CH₃-coumarin-3-acetate (AMCA). FAM, HEX, TET, JOE, ROX, and TAMRA are marketed by Perkin Elmer, Applied Biosystems Division (Foster City, Calif.). Texas Red and many other suitable compounds are marketed by Molecular Probes (Eugene, Oreg.). Examples of chemiluminescent and bioluminescent compounds that may be suitable for use as the energy donor include luminol (aminophthalhydrazide) and derivatives, and Luciferases.

While in most embodiments it will be preferred that the detectable label be a light-emitting label and the masking group be a quencher, such as a quenching chromophore, other detectable labels and masking groups are possible. For example, the label may be an enzyme and the masking group an inhibitor of said enzyme. When the enzyme and inhibitor are in sufficiently close proximity to interact, the inhibitor is able to inhibit the activity of the enzyme. On cleavage or denaturation of the probe, the enzyme and inhibitor are separated and no longer able to interact, such that the enzyme is rendered active. A wide variety of enzymes capable of catalysing a reaction resulting in the production of a detectable product and inhibitors of the activity of such enzyme are well known to the skilled artisan, such as β-galactosidase and horseradish peroxidase.

In a further aspect the present invention provides a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridized to the first nucleic acid molecule, the second nucleic acid molecule comprising at least one copy of the target nucleic acid sequence, or a detection sequence, or both.

In one embodiment, at least one nucleic acid molecule of the dimeric probe is circular.

In another embodiment, the dimeric probe is linear. Preferably, the target binding domain is located at the 5′OH terminus, the 3′OH terminus, or both termini, of the first strand.

Preferably either or both of the first and second nucleic acid molecules are not susceptible to exonucleolytic activity in the absence of cleavage of the nuclease cleavage element, more preferably the 5′OH terminus, the 3′OH terminus, or both termini of either or both nucleic acid molecules contains a blocking group capable of blocking exonuclease activity.

In one embodiment, the nuclease cleavage element is one strand (the monomeric nucleic acid sequence component) of a restriction endonuclease recognition site, whereby when bound to target sequence, the target binding domain forms a restriction endonuclease recognition site.

In one embodiment, when said target nucleic acid is DNA, said nuclease cleavage element comprises RNA.

Preferably the detectable label is a fluorophore and said masking group is a quencher capable of quenching the fluorescence of said fluorophore when in sufficiently close proximity.

In a particularly preferred embodiment, the invention provides a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule also carrying a fluorophore, the probe comprising a second nucleic acid molecule hybridized to the first nucleic acid molecule, the second nucleic acid molecule comprising at least one copy of the target nucleic acid sequence and carrying a quencher.

Preferably, said fluorophore is positioned 5′ to the cleavage element of the first nucleic acid molecule.

Preferably, said quencher is positioned 5′ to the at least one copy of the target nucleic acid sequence of the second nucleic acid molecule.

More preferably, said quencher is positioned 5′ to the nuclease cleavage element within the at least one copy of the target nucleic acid sequence of the second nucleic acid molecule.

Still more preferably, when there is more than one copy of the target nucleic acid sequence of the second nucleic acid molecule, the quencher is positioned 5′ to the nuclease cleavage elements of the all of the more than one copy of the target nucleic acid sequence.

In one preferred embodiment, the present invention provides a dimeric polynucleotide probe for detecting a target nucleic acid, said dimeric probe comprising a first nucleic acid molecule hybridised to a second nucleic acid molecule,

wherein the first nucleic acid molecule additionally contains a target-binding domain, said target-binding domain comprising nucleic acid sequence complementary to or capable of hybridising to said target nucleic acid;

and wherein the target-binding region contains a nuclease cleavage element capable on hybridising with a sequence corresponding to said target nucleic acid of forming a region subject to cleavage by a nuclease,

and wherein the first nucleic acid molecule contains a detectable label the signal of which is diminished or rendered undetectable when in sufficiently close proximity to a masking group,

and wherein the second nucleic acid molecule contains at least one region comprising nucleic acid sequence corresponding to the target nucleic acid or a sequence capable of hybridising to the target-binding domain of the first nucleic acid molecule and that when single-stranded is capable on hybridising to the target-binding domain of a first nucleic acid molecule of forming a region subject to cleavage by a nuclease,

and wherein the second nucleic acid molecule contains a masking group capable of diminishing or rendering undetectable the signal of said label when said first nucleic acid molecule is intact,

and wherein when the dimeric probe is intact, the detectable label and the masking group are in sufficiently close proximity that the masking group diminishes or renders undetectable the signal of the detectable label.

Preferably, blockers are used to prevent exonuclease activity on the un-triggered probe. These could be any modified form of DNA including amino linkage, thiol linkage, 3′-3′ linkage, 5′-5′ linkage, nucleoside analogues, spacers or 5′ or 3′ terminal modifications including dephosphorylation. In addition, the termini may be blocked with short complementary strands of modified DNA, including PNA, or be blocked by binding proteins.

Various configurations of dimeric probe allow variations in the method to detect target nucleic acid to be employed.

Thus, in another aspect, the invention provides a method for detecting a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule carrying a quencher and comprising at least one copy of the target nucleic acid sequence, the probe comprising a second nucleic acid molecule hybridized to the first nucleic acid molecule, the second nucleic acid molecule carrying a fluorophore,
c) contacting the sample with an excess of the probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a fluorescent signal and exposing the at least one copy of the target nucleic acid sequence on the first nucleic acid molecule,
f) allowing the target binding domain of the excess probe to bind the exposed at least one copy of the target nucleic acid sequence on the first nucleic acid molecule,
g) repeating steps d) and e) to amplify the fluorescent signal and expose additional copies of the at least one copy of the target nucleic acid sequence, and
h) repeating steps f) and g) to amplify the fluorescent signal to a desired level,
i) detecting or measuring the fluorescent signal,
wherein an increase in signal is indicative of the presence of the target nucleic acid in the sample.

In various embodiments, the steps a) to h) are carried out sequentially or simultaneously.

The invention also provides a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule carrying a quencher and comprising at least one copy of the target nucleic acid sequence, the probe comprising a second nucleic acid molecule hybridized to the first nucleic acid molecule, the second nucleic acid molecule carrying a fluorophore.

The present invention also provides methods utilising a probe that in essence is a single element of the RNCR probes described herein. These methods are referred to herein as RNAse-Mediated Detection (RMD), and can be used for DNA target detection where there is a sufficiently high number of target molecules such that geometric amplification is not required. Alternatively, it can be used in conjunction with any DNA amplification method, in addition to the detection methods described herein.

The method again uses FRET to generate a discriminatory fluorescent signal, but differs from the dual-labelled TaqMan probes in that it uses an RNA probe and a ribonuclease H.

TaqMan DNA probes are incompatible with the isothermal DNA amplifications systems and are of limited use on static, post-amplification DNA samples. On binding to the DNA they remain intact (and hence quenched) unless cleaved by a nuclease. Signal could be obtained using a TaqMan probe with an endonuclease site, but such a method is destructive to the target as well as the probe and so one target can produce only one unquenched fluorophore. The non-destructive nature of ribonuclease H enzymes to the DNA strand of RNA/DNA hybrids means that use of an RNA probe will leave the target DNA intact. Moreover, the action of the enzyme completely hydrolyses the annealed probe and so allows a new probe to bind. In essence, the DNA strand merely acts as a catalyst for the enzyme-mediated cleavage of the probe.

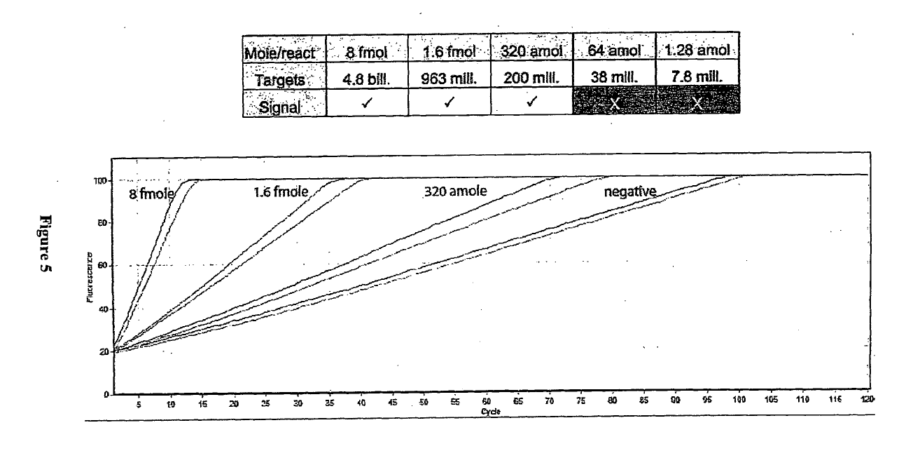

Hence, with sufficient probe, signal strength will increase in a linear fashion over time. The method is highly sensitive. The use of such a system to detect as few as 320 amoles (3.2×10⁻¹⁶) of target sequence (approximately 200 million molecules) is described herein in Example 4. See the Table of FIG. 5 herein. Such sensitivity levels are exceptionally good for an isothermal detection method, and when combined with isothermal amplification provide a powerful detection system.

Accordingly, in a further aspect the invention provides a method for detecting a target DNA in a sample, the method comprising the steps

a) providing a sample containing a target DNA sequence,
b) providing a single stranded RNA probe carrying a detectable label and a masking group, the probe comprising a target binding domain,
c) contacting the sample with the probe so the target binding domain binds the target DNA sequence,
d) contacting the sample with a nuclease to degrade bound probe and separate the detectable label from the masking group, thereby generating a signal,
e) detecting or measuring the signal,
wherein an increase in signal as compared to the signal of the intact probe is indicative of the presence of the target nucleic acid in the sample.

Preferably, the detectable label is a fluorophore and the masking group is a quencher.

Preferably, the nuclease is ribonuclease H(RNAse H or an agent having RNAse H activity. As used herein, an “agent having ribonuclease H activity” includes ribonuclease H, variants and functional equivalents thereof, whereby functional equivalents are any compound, moiety or enzyme that has nucleolytic activity against the RNA component of an RNA:DNA hybrid, yet has no nucleolytic activity against the DNA component of an RNA:DNA hybrid.

It will be appreciated that the RMD method can also be used in conjunction with the NCR, RNCR and PNCR methods described herein. Example 5 herein describes the use of the RMD method in combination with RNCR. Such combinations allow for unlabelled (and thus lower cost) NCR probes and RNCR probes to be manufactured and used. With this embodiment of the methods of the invention, the signal is generated by an RMD probe which can be kept generic, irrespective of target sequence to be detected. Under ideal conditions, signal generation is enhanced by the combined geometric tripling of the RNCR probe and the linear signal amplification of the RMD method.

It will be apparent that in such combined embodiments, when present in the dimeric probes of the invention the detection sequence as used herein is a sequence able to hybridise to the RMD probe.

Thus in a preferred embodiment, the present invention provides a method for detecting target nucleic acid in a sample, said method comprising the steps of:

a) contacting a sample comprising target nucleic acid with a first probe and a second probe,
wherein said first probe is a dimeric probe comprising a first nucleic acid molecule and a second nucleic acid molecule,
and wherein each of said first and second nucleic acid molecules contain at least one duplex-forming region substantially complementary to or capable of hybridising one to the other and which when hybridised form at least one double-stranded region,
and wherein the first nucleic acid molecule additionally contains a target-binding region, said target-binding region containing a nuclease cleavage element capable on hybridising with a sequence corresponding to said target nucleic acid of forming a region subject to cleavage by a nuclease,
and wherein the second nucleic acid molecule contains at least one region capable of hybridising to the second probe and that when single-stranded is capable on hybridising to a second probe of forming a region subject to cleavage by a ribonuclease H activity,
   thereby to form a target nucleic acid:probe hybrid containing a region subject to cleavage by a nuclease;

b) contacting the mixture of a) with a nuclease in an amount sufficient to selectively cleave the target nucleic acid:probe hybrid;

c) contacting the mixture of b) with a strand-separating activity in an amount sufficient to dissociate the first and second nucleic acid molecules or degrade the first nucleic acid molecule of the first probe;

d) contacting the mixture of c) with an amount of ribonuclease H sufficient to cleave the second probe and cause sufficient separation of the label and the masking group to diminish or prevent the masking activity of said masking group;

e) detecting or measuring the separation of label and masking group by detecting an increase in the signal of the label as compared to the signal of the intact second probe;

wherein an increase in signal is indicative of the presence of said target nucleic acid in the sample.

When unlabelled first probe is used, signal amplification is triggered only when the reaction components—the target nucleic acid, the first probe, the second probe, the nuclease, and the strand-separating activity—are all present. In one embodiment, the target nucleic acid may be contacted with a composition containing the first probe, the second probe, the nuclease, and the strand-separating activity. Advantageously, this minimises the opportunities for introducing contamination when the method is performed in a closed system. Preferably, step b), step c) and step d) are performed contemporaneously, more preferably, step a), step b), step c) and step d) are performed contemporaneously.

The region of the second nucleic acid molecule capable of hybridising to the second probe (referred to elsewhere herein as a detection sequence) may contain nucleic acid sequence corresponding to the target nucleic acid or a sequence capable of hybridising to the target-binding region of the first nucleic acid molecule. In this embodiment, the second probe is therefore able to bind to either the target nucleic acid, or to the second nucleic acid molecule of the first probe. This potentially increases the number of target sites for binding of the second probe, leading to increased signal amplification.

While the above method allows for the production and use of unlabelled NCR, RNCR, and PNCR probes, thereby reducing the cost of the method, it will be apparent to the skilled artisan that the first probe can itself be labelled. Such embodiments are described herein. It will be apparent that when labelled first probe is used, signal amplification may be triggered in the absence of second probe.

In a further aspect, the invention provides a single stranded RNA probe comprising a fluorophore, a quencher and a target binding domain. For convenience, such probes are referred to herein as RMD probes.

The invention recognises that additional copies of target sequence and detection sequence can be generated using a strand-displacement polymerase and a continuous template, for example, a circular probe molecule. Here, the polymerase both separates the first and second nucleic acid molecules of the dimeric probe, and generates a reverse complement of the template nucleic acid molecule. By configuring the sequence on the template molecule appropriately, the reverse complement generated by the polymerase contains additional copies of target sequence (thereby allowing additional dimeric probe to bind and trigger further displacement/polymerisation reactions) and of detection sequence able to be bound by a detection probe.

Accordingly, in another aspect, the invention provides a method for detecting a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second, circular nucleic acid molecule hybridized to the first nucleic acid molecule, the second nucleic acid molecule comprising at least one copy of a sequence that is the reverse complement of the target nucleic acid sequence and at least one copy of a sequence that is the reverse complement of a detection sequence,
c) contacting the sample with an excess of the dimeric probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a reverse complement of the second nucleic acid molecule containing at least one copy of the target nucleic acid sequence and at least one copy of the detection sequence,
f) allowing the target binding domain of the excess probe to bind the exposed at least one copy of the target nucleic acid sequence on the reverse complement of the second nucleic acid molecule,
g) repeating steps d), e), and f) to expose additional copies of the at least one copy of the target nucleic acid sequence, or the detection sequence, or both, and
h) contacting the sample with a second probe that binds the detection sequence, the second probe carrying a detectable label,
i) detecting or measuring the signal of the detectable label,
wherein an increase in signal is indicative of the presence of the target nucleic acid in the sample.

In various embodiments, the steps a) to h) are carried out sequentially or simultaneously.

Preferably, the second probe is a single stranded RNA probe comprising a fluorophore, a quencher and a detection sequence binding domain, more preferably the second probe is an RMD probe as described herein.

In another aspect the invention provides a method for increasing the number of copies of a target nucleic acid in a sample, the method comprising the steps

a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridized to the first nucleic acid molecule, the second nucleic acid molecule comprising at least one copy of the target nucleic acid sequence, or a detection sequence, or both,
c) contacting the sample with an excess of the probe so the target binding domain binds the target nucleic acid sequence,
d) contacting the sample with a first nuclease to cleave the nuclease cleavage element or degrade the target binding domain,
e) separating the first and second nucleic acid molecules of the dimeric probe to expose the at least one copy of the target nucleic acid sequence on the second nucleic acid molecule, or the detection sequence, or both,
f) allowing the target binding domain of the excess probe to bind the exposed at least one copy of the target nucleic acid sequence on the second nucleic acid molecule,
g) repeating steps d) and e) to expose additional copies of the at least one copy of the target nucleic acid sequence, or the detection sequence, or both, and
h) repeating steps f) and g) to expose a desired amount of the at least one copy of the target nucleic acid sequence, or the detection sequence, or both.

It will be apparent to the skilled addressee that the nature of the method allows for the amplification of binding sites for the target binding domain of the dimeric probe, without the contemporaneous synthesis of target nucleic acid. The number of copies of target sequence available for binding by the target binding domain is increased as a result of the separation of first and second molecules of the dimeric probe.

In still a further aspect, the invention provides a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second, circular nucleic acid molecule hybridized to the first nucleic acid molecule, the second nucleic acid molecule comprising at least one copy of a sequence that is the reverse complement of the target nucleic acid sequence and at least one copy of a sequence that is the reverse complement of a detection sequence.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the probe comprising a target binding domain and at least one copy of the target nucleic acid sequence.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the first nucleic acid molecule comprising a target binding domain and the second nucleic molecule comprising at least one copy of the target nucleic acid sequence.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the probe comprising a target binding domain and at least one copy of the target nucleic acid sequence, wherein the at least one copy of the target nucleic acid sequence is available for binding by the target binding domain of another copy of the probe when the first and second nucleic acid molecules of the dimeric probe are separated.

In yet a further aspect, the invention provides a dimeric polynucleotide probe for the detection of a target nucleic acid sequence, the probe comprising a first nucleic acid molecule and a second nucleic acid molecule hybridized to the first nucleic acid molecule, the probe comprising a target binding domain and at least one copy of the target nucleic acid sequence, wherein the at least one copy of the target nucleic acid sequence is masked when the dimeric probe is intact and is available for binding by the target binding domain of another copy of the probe when the first and second nucleic acid molecules of the dimeric probe are separated.

Also provided are methods and probes as described above and herein, with reference to the examples and figures.

In another aspect, the present invention provides a composition containing a probe of the invention, together with one or more additives, buffers, excipients, or stabilisers.

Preferably, the composition additionally contains one or more of the group comprising:
a nuclease;
a strand-separating activity;
a compound, co-factor or co-enzyme to activate or augment the activity of an agent having nuclease activity;
a compound, co-factor or co-enzyme to activate or augment the strand-separating activity.

In another aspect, the present invention provides a kit for detecting target nucleic acid in a sample, said kit comprising a quantity of dimeric probe of the invention, a quantity of a nuclease, and a quantity of a strand-separating activity, together with instructions for contacting the probe, the nuclease, and the strand-separating activity with the sample.

In a further aspect, the present invention provides a kit for detecting target nucleic acid in a sample, said kit comprising a quantity of probe of the invention, a quantity of a ribonuclease H, together with instructions for contacting the probe and the ribonuclease H with the sample.

In one embodiment, the kit contains a quantity of dimeric probe of the invention, a quantity of an RMD probe of the invention, a quantity of a nuclease, a quantity of a strand-separating activity, together with instructions for contacting the probe, the nuclease, and the agent having strand-separating activity with the sample.

Kits containing the materials necessary for carrying out the methods of the invention can be assembled to facilitate handling and foster standardization. Typically the kit would include the dimeric probe, the nuclease, and the exonuclease, necessary buffers, and one or more standards. The standards can be target nucleic acid, nuclease or exonuclease substrates, or data (empirical) in printed or electronic form necessary for the calibration needed to carry out the methods of the invention. Materials to be included in the kit, and the form in which the kit components are provided, may vary depending on the ultimate purpose. For example, the use of solid phase technologies (for example, but not limited to the well-known “dipstick” technologies), where reaction components such as the dimeric probe, the nuclease and exonuclease activities are deposited on a solid substrate which is then contacted with sample to be analysed for the presence of target nucleic acid, readily allow the analysis of samples in situations where laboratory facilities are not available, for example in the field.

It will be appreciated that a kit may comprise a single species of probe and is thereby able to indicate the presence of a single species of target nucleic acid, or may comprise multiple species of probe, where the presence of multiple species of target nucleic acid can be indicated. In the latter embodiment it may be desirable to have the different species of probe differentially labelled, so that the identity of the one or more species of target nucleic acid present can be determined. However, in other cases identification of the specific target nucleic acid species is not required, wherein it would not be necessary to differentially label the various species of probe.

It will also be apparent that the first and second nucleic acid molecules that comprise the dimeric probes of the invention may be provided separately, to be hybridised before use in the methods of the invention. In such circumstances, instructions for the correct method to hybridise the molecules, including the appropriate relative amounts thereof, should also be provided.

It will also be appreciated that the materials present in the kit can be chosen so as to enable qualitative, semi-quantitative, or quantitative evaluation of the target nucleic acid present in the sample. For example, the kit can give a binary (yes/no) indication of whether the one or more species of target nucleic acid is present in the sample. In another example, the indication may be semi-quantitative, for example, by giving three levels of signal—high, low, and no signal. Depending on the label, these levels could for example be shades of the same colour, wherein a darker shade indicates a high level of target nucleic acid, a medium shade indicates a low level of target nucleic acid, and a light shade or no colour indicates no target nucleic acid present. The kit may also provide for the quantitative analysis of target nucleic acid, for example by measurement, including real-time measurement, of the production of signal. An example of such quantitative measurement of target nucleic acid is presented herein in the Examples.

The methods and probes of the invention have broad application in all areas where the presence or amount of a particular nucleic acid is to be determined. Non-limiting examples of the uses of NCR, RNCR, PNCR and RMD include:
(i) the detection of microbial agents, including pathogenic bacteria and viruses, in both the field of medicine and in the detection of agents of bioterrorism;
(ii) the detection of parasitic diseases, for example Malaria, Trypanosomes, Leishmania;
(iii) the detection, discrimination, or quantification of human DNA for forensic purposes or the detection, discrimination, or quantification of animal or plant DNA for veterinary or agricultural purposes;
(iv) the detection of microbial, plant or insect pests for biosecurity;
(v) the detection of genetically modified organisms;
(vi) the detection of specific genetic alleles or polymorphisms.

The term “comprising” as used in this specification and claims means “consisting at least in part of”, that is to say when interpreting statements in this specification and claims which include the term, the features, prefaced by that term in each statement, all need to be present but other features can also be present.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

It will be appreciated that variants of nucleic acids, for example, of target nucleic acids or the dimeric probes of the invention, can by utilized in the methods of the present invention.

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the inventive polypeptides and polynucleotides possess biological activities that are the same or similar to those of the inventive polypeptides or polynucleotides. The term “variant” with reference to polynucleotides and polypeptides encompasses all forms of polynucleotides and polypeptides as defined herein.

Variant polynucleotide sequences preferably exhibit at least 50%, more preferably at least 70%, more preferably at least 80%, more preferably at least 90%, more preferably at least 95%, more preferably at least 98%, and most preferably at least 99% identity to a sequence of the present invention. Identity is found over a comparison window of at least 5 nucleotide positions, preferably at least 10 nucleotide positions, preferably at least 20 nucleotide positions, preferably at least 50 nucleotide positions, more preferably at least 100 nucleotide positions, and most preferably over the entire length of a polynucleotide of the invention.

Polynucleotide sequence identity can be determined in the following manner. The subject polynucleotide sequence is compared to a candidate polynucleotide sequence using BLASTN (from the BLAST suite of programs, version 2.2.5 [November 2002]) in b12seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of b12seq may be utilized.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Aligmnent. Computer Applications in the Biosciences 10, 227-235.

Use of BLASTN as described above is preferred for use in the determination of sequence identity for polynucleotide variants according to the present invention.

Alternatively, variant polynucleotides of the present invention hybridize to the polynucleotide sequences disclosed herein, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing). Tm for polynucleotide molecules greater than about 100 bases can be calculated by the formula Tm=81.5+0.41% (G+C−log(Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide molecules of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available b12seq program from the BLAST suite of programs (version 2.2.5 [November 2002]) from NCBI (ftp://ftp.ncbi.nih.gov/blast/) via the tblastx algorithm as previously described.

The variant polynucleotide sequences of the invention may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

To identify the polynucleotide variants most likely to be functional equivalents of the disclosed sequences, several further computer based approaches are known to those skilled in the art.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

A “fragment” of a polynucleotide sequence provided herein is a subsequence of contiguous nucleotides that is at least 5 nucleotides in length. The fragments of the invention comprise at least 5 nucleotides, preferably at least 10 nucleotides, preferably at least 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide of the invention.

The term “primer” refers to a short polynucleotide, usually having a free 3′OH group, that is hybridized to a template and used for priming polymerization of a polynucleotide complementary to the target.

Methods for assembling and manipulating genetic constructs and vectors, together with the use of enzymes commonly employed in molecular biological techniques, including nucleases such as ribonucleases, exonucleases and restriction endonucleases, polymerases, ligases and the like, are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

Various aspects of the invention will now be illustrated in a non-limiting way by reference to the following examples. The following examples describe the use of fluorescent labels (fluorophores) and quenchers. However, this is primarily for the sake of convenience and is not intended to limit the application in any way. As described above, it will be apparent that other labels and masking groups can be used in the methods of the invention.

### EXAMPLES

### Example 1

### A Linear Dimeric NCR Probe

This example describes the elements of one embodiment of a Nuclease Chain Reaction (NCR) probe, and the steps in the chain reaction leading to signal amplification. It should be noted that the order of the elements can differ from that described herein and shown in the accompanying figures.

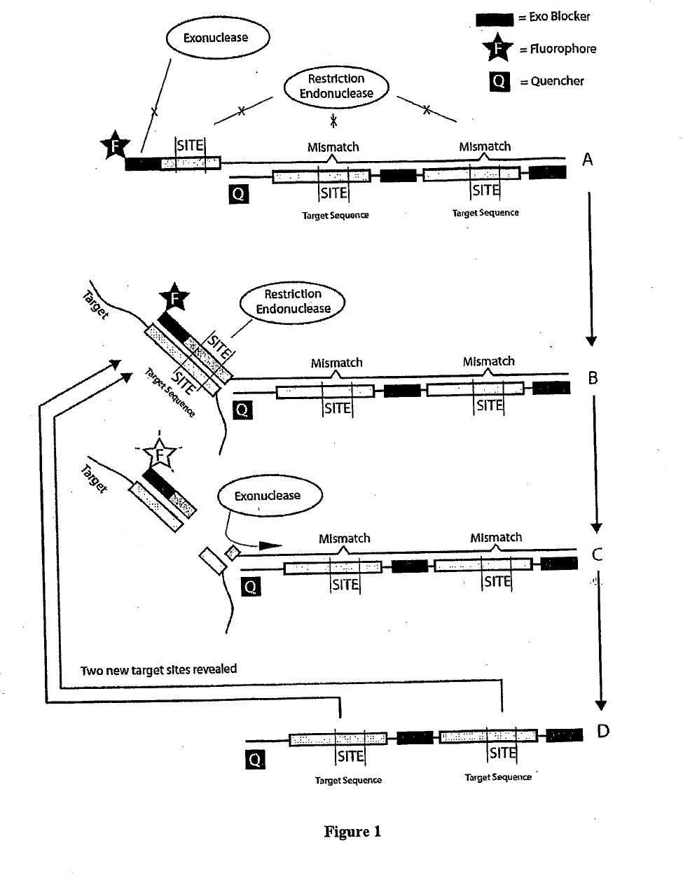

The configuration of a linear, double-stranded polynucleotide NCR probe is shown in FIG. 1, panel A. In this embodiment of the probe, the single-stranded extension is on the 5′ end of one of the DNA strands and this position is appropriate for a 5′-3′ exonuclease. See FIG. 1, Panel A.

Blockers (referred to in FIG. 1 as “Exo Blocker”) are used to prevent exonuclease activity on the un-triggered probe. Any modified form of nucleic acid including amino linkage, thiol linkage, 3′-3′ linkage, 5′-5′ linkage, nucleoside analogues, spacers or 5′ or 3′ terminal modifications including dephosphorylation are used.

The linear probe contains multiple copies of a restriction endonuclease recognition site (referred to in FIG. 1 as “SITE”).

Internal modifications of the lower (second) polynucleotide are included to reduce exonuclease activity between the cleavage sites and where possible, to act as a clamp to maintain the double-stranded configuration. These modifications do not have a significant effect on the ability of the upper polynucleotide to be degraded by the exonuclease. Here, a peptide nucleic acid (PNA) region is included, and would reduce exonuclease activity but not completely block it (Slaitas et al, 2003). The use of PNA's will also help to stabilise the double-stranded portion of the probe. Stabilization is essential to prevent denaturation of the probe and hence false triggering by revealing the lower (second) strand.

A fluorophore and a quencher are included in the molecule to enable FRET-based detection (Livak et al, 1998). These elements can be placed in a number of possible positions as long as the two are separated from each other by the action of either the endonuclease or the exonuclease. In this embodiment, the quencher and/or the fluorophore are positioned on also assist as exonuclease blockers on the strand termini.

In the native, un-triggered configuration, the probe is resistant to both the endonuclease(s) and exonuclease(s). The probe is protected from the exonuclease the blocking elements. The endonuclease will have no activity on the single-stranded portion of the molecule, as its substrate is a double-stranded recognition site. Similarly, cleavage is prevented on the double-stranded sites by mismatched sequences on the upper (first) strand located within the two cleavage sites, thereby destroying the recognition site.

The binding of probe to the target sequence is shown in FIG. 1, panel B. When the single-stranded target-binding region of the probe binds to a complementary target sequence, a double-stranded restriction endonuclease site is created allowing the DNA to be cut. In so doing, the enzyme exposes two unmodified strand ends that are suitable targets for the exonuclease. During this digestion, the fluorophore and the quencher become physically separated giving an increase in overall levels of fluorescence.

The degradation of the upper polynucleotide by the exonuclease is shown in FIG. 1, panel C. In this embodiment, unmodified, phosphorylated 5′ termini are created by the endonuclease cleavage event. Here, the exonuclease degrades the upper polynucleotide from left to right (5′-3′). The cleaved short portion of lower polynucleotide will also be degraded from right to left as far as the blocker. Only a 3′ terminus is revealed on the lower (second) strand of the probe itself and so no hydrolysis can occur.

Two new target sequences are exposed by the exonucleolytic degradation of the upper polynucleotide, as shown in FIG. 1, panel D. With the upper polynucleotide removed, two new target sequences are revealed on the lower strand. In this embodiment, these have the same sequence as the original target sequence. These two sites can now become targets for the single-stranded target-binding regions of two new probes.

With the positioning of the quencher as shown in FIG. 1, hybridisation of the newly exposed intrinsic targets to new probes will bring about a short term quenching of the probes' fluorophores. Once the hybrid molecule is cleaved with the restriction endonuclease, the exonuclease will hydrolyse the nucleotide bases up to the quencher and so release it.

At each reaction iteration, the number of targets doubles, leading to a geometric amplification of signal.

### Example 2

### A Linear Dimeric RNCR Probe

This example describes the elements of an alternative conformation of an NCR probe, which comprises a RNA:DNA chimeric probe and is referred to as an RNCR probe. The steps in the chain reaction leading to signal amplification are also described. Again it should be noted that the order of the elements can differ from that described herein and shown in the accompanying figures.

This embodiment is known as Ribonuclease-NCR(RNCR). Ribonuclease H enzymes have endonuclease activity on the RNA strand of RNA/DNA hybrid molecules. They have no activity on DNA/DNA or RNA/RNA molecules, nor do they hydrolyse single-stranded DNA or RNA.

One advantage of this embodiment is that it is non-destructive to the target sequence. Therefore, the RNCR can follow a more rapid geometric progression than can NCR. Unlike NCR, PCR or LCR, RNCR performs a tripling of targets at each reaction iteration, rather than a doubling.

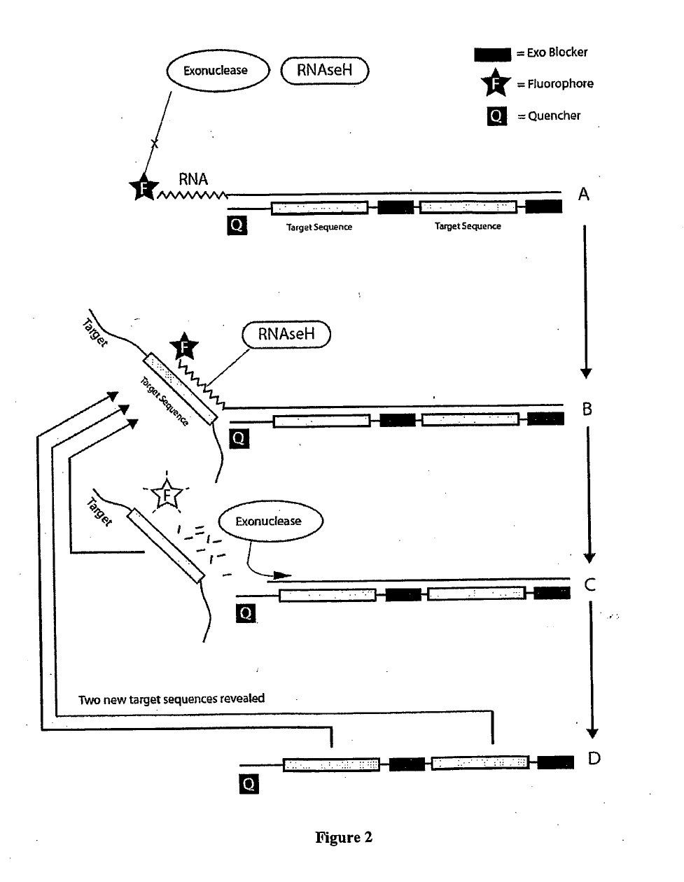

The configuration of the double-stranded polynucleotide RNCR probe is shown in FIG. 2, panel A. In this embodiment, the single-stranded extension containing the target-binding region is RNA rather than DNA. Because this configuration does not use site specific-endonucleases, any target sequence can be chosen.

The other elements such as the blockers, fluorophore and quencher are of a type and are positioned in a similar manner as those described for NCR.

The binding of probe to the target sequence is shown in FIG. 2, panel B. When the RNA region of the probe binds to a complementary DNA sequence, a hybrid RNA/DNA region is created. Once the hybrid is formed, it is available for hydrolysis by the ribonuclease H.

The degradation of the RNA region of the probe by ribonuclease H is shown in FIG. 2, panel C. With removal of the RNA portion of the probe, DNA termini are exposed that are suitable substrates for the exonuclease. The exonuclease hydrolysis progresses as in NCR as discussed in Example 1.

Two new target sequences are exposed by the exonucleolytic degradation of the upper polynucleotide, as shown in FIG. 2, panel D. The exonuclease reveals two new target sites providing targets for two new probes. Because the original target site is revealed and not destroyed by the activity of RNAse H on the target:probe hybrid, there are now three targets available for the next iteration of the reaction.

### Example 3

### A Linear Dimeric PNCR Probe

This example describes the elements of an alternative conformation of an NCR probe, designed for use in a method which uses a DNA polymerase with displacement activity to expose the intrinsic target sequences. This method variant is known as Polymerase Nuclease Chain Reaction (PNCR). The steps in the chain reaction leading to signal amplification are also described. Again it should be noted that the order of the elements can differ from that described herein and shown in the accompanying figures.

One advantage of this embodiment is that by avoiding exonucleases, fewer protective elements are required to prevent degradation of the probe and the target nucleic acid.

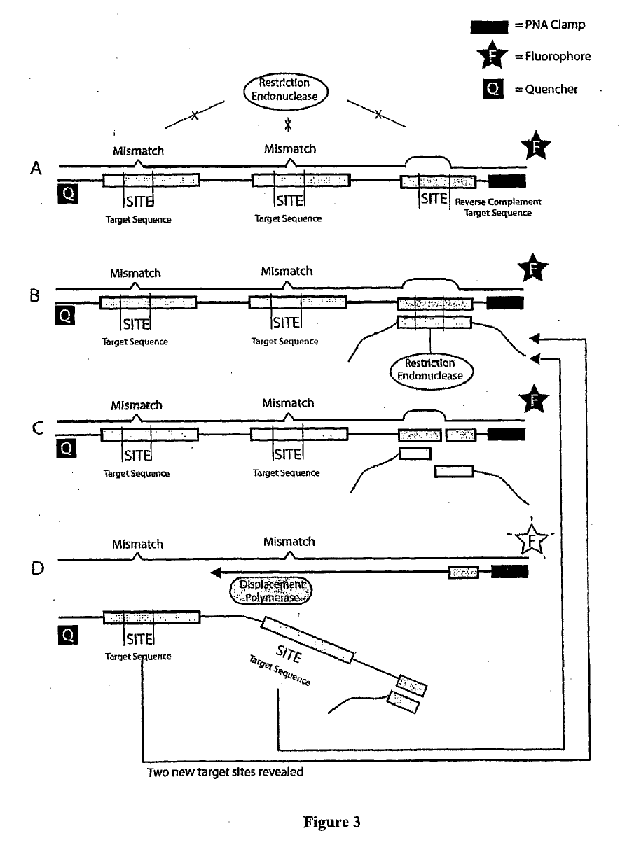

The configuration of the double-stranded polynucleotide PNCR probe is shown in FIG. 3, panel A. In this embodiment, the region of the probe which is reverse complement to the target has a second mismatched site held in place by a terminal peptide nucleic acid (PNA) clamp.

The mismatched region is sufficiently unfavourable for binding that invasion can occur from a perfectly matched target. More mismatched nucleotides can be included in the upper strand to create a bulge-loop.

Mismatches on the other restriction endonuclease sites prevent enzyme activity within the double-stranded region of the probe. The fluorophore and quencher are of a type and are positioned in a similar manner as for NCR as described above in Example 1.

The binding of the probe to the target sequence is shown in FIG. 3, panel B. When the mismatched region of the probe binds to a complementary DNA sequence, a double-stranded site is created. The number of nucleotides unpaired between the upper strand and the lower strand can be increased at this point by strand replacement.

The cleavage of the hybridised region by restriction endonuclease is shown in FIG. 3, panel C. The double-stranded region of the target:probe hybrid is cleaved. As the site is proximal to one end of the hybridized DNA, the number of hydrogen bonds anchoring the target DNA at the probe end most proximal to the fluorophore is sufficiently low to permit the release of this DNA. Nucleotides on the upper strand previously displaced by the target DNA re-anneal with lower strand thereby creating a primer for a DNA polymerase.

The polymerase-mediated displacement of the lower strand is shown in FIG. 3, panel D. The displacement DNA polymerase extends the primer to generate a new reverse-complement of the upper strand while displacing the lower strand. This displacement reveals two, new target present in the lower strand. These are now available for further binding as shown in panel B.

At each iteration, the target number doubles.

### Example 4

### A Single Element RNCR Probe

This example describes a probe for use in a simplified detection system that in essence is a single element of the RNCR probe. The method, referred to herein as RNAse-Mediated Detection (RMD), can be used for DNA target detection where there is a sufficiently high number of target molecules and therefore, geometric amplification is not required. Alternatively, it can be used in conjunction with any DNA amplification method, in addition to the detection methods described herein.

The method again uses FRET to generate a discriminatory fluorescent signal but differs from the dual-labelled TaqMan probes in that it uses an RNA probe and a ribonuclease H. TaqMan DNA probes are incompatible with the isothermal DNA amplifications systems (see above) and they are limited in their use on static, post-amplification DNA samples. On binding to the DNA they remain intact (and hence quenched) unless cleaved by a nuclease. Signal could be obtained using a TaqMan probe with an endonuclease site, but such a method is destructive to the target as well as the probe and so one target can produce only one unquenched fluorophore.

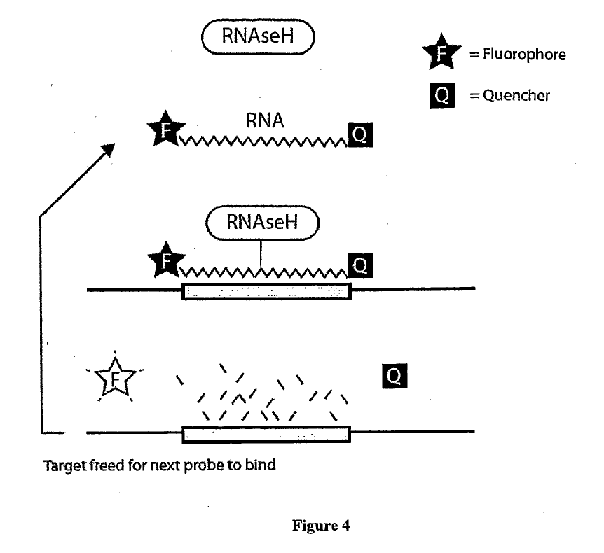

In RMD, the non-destructive nature of ribonuclease H enzymes with respect to the DNA strand of RNA/DNA hybrids means that an RNA probe will leave the target intact (FIG. 4). Moreover, the action of the enzyme completely hydrolyses the annealed probe and so allows a new probe to bind. In essence, the DNA strand merely acts as a catalyst for the enzyme-mediated cleavage of the probe.

Hence, with sufficient probe, signal strength will increase in a linear fashion over time.

An RMD detection reaction was conducted as follows. All reactions were carried out in the following buffer: 20 mM HEPES (pH 7.6), 50 mM KCl, 10 mM MgCl2 and 1 mM dithiothreitol (DTT).

A 25 μl reaction was used containing 0.2 U of RNAseH and 50 pmole of an RMD probe (5′FAM-UUCAAGCGAUUCUCCU-TAMRA-3′ [SEQ ID. NO. 1]). A serial dilution was made of a synthetic target oligonucleotide (5′ AGGCTGAGGCAGG AGAATCGCTTGAACCAAGGAGGC 3′ [SEQ ID. NO. 2]) from a 10 μM stock.

Reactions were monitored for FAM fluorescence at 30 second intervals in a Corbett RotorGene Real-time PCR machine.

This experiment shows that such a system can easily detect 320 amoles (3.2×10⁻¹⁶) of target sequence—approximately 200 million molecules (see Table, FIG. 5). Such sensitivity levels for an isothermal detection method are exceptionally good and when combined with isothermal amplification, will provide a powerful detection system.

### Example 5

### The Use of RMD in Combination with RNCR

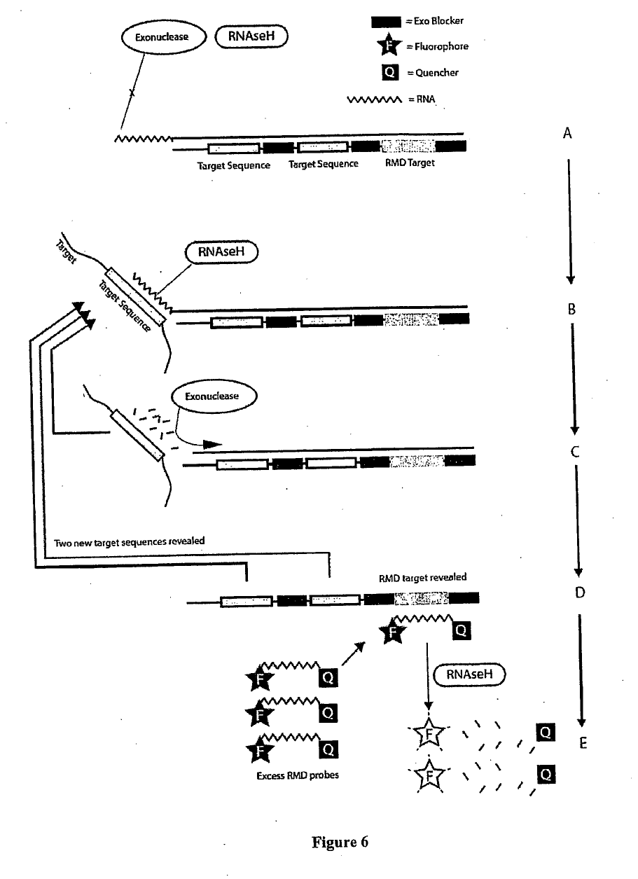

This example describes a combined method using a combination of RMD and RNCR. Here, an unlabelled (and hence lower cost) RNCR probe is used, and the signal is generated by an RMD probe. A single species of RMD probe can be used with a variety of target-specific RNCR probes by incorporating the same RMD target sequence into each RNCR probe (see FIG. 6, panel A). Under ideal conditions, signal generation is enhanced by the combined geometric tripling of the RNCR probe and the linear signal amplification of the RMD method.

As described in Example 2 above, the RNCR probe binds target sequence (FIG. 6, panel B), and the target:probe hybrid is cleaved by RNAse H. This leads to degradation of the upper polynucleotide (FIG. 6, panel C), revealing the intrinsic target sequences and the RMD target sequence. RMD probe binds to the RMD target sequence, forming an RNA:DNA hybrid which is cleaved by RNAse H and signal is generated (FIG. 6, panel D).

### Example 6

### A Circular Dimeric PNCR Probe and Rolling Circle PNCR

This example describes an alternative embodiment of the invention where a circular dimeric probe is used in a combination of the PNCR and the RMD detection methods. This method uses a displacement polymerase and a rolling-circle method of replication and so is named Rolling Circle PNCR (RC-PNCR)

The configuration of the dimeric polynucleotide probe is shown in FIG. 7, panel A. Present in the circular polynucleotide of the probe is a reverse complement copy of the target. This is partially obscured by a second short polynucleotide of the dimeric probe. This second nucleic acid molecule has a mismatch in the restriction enzyme recognition site to prevent the molecule being cut.

The target-binding region of the probe is present in a single stranded region of the duplex. Here, this is achieved by incorporating non-complementary bases into the circular molecule. If the probe binds to the wrong site (non-specific binding) there is a high probability that it will not generate a restriction site.

Also on the circular polynucleotide is an exact copy of the RMD probe sequence. This is not the reverse complement and so the dual labeled RMD probe cannot bind to it. The choice of sequence for the RMD probe is essentially random but chosen to minimize secondary structure artifacts.

The binding of the RC-PNCR probe to the target nucleic acid is shown in FIG. 7, panel B. When the probe anneals to the chromosomal DNA, a restriction site is created. This brings about a small region of strand exchange adjacent to the restriction site. This region will become the primer for the polymerase.

Cleavage of the restriction site by endonuclease is shown in FIG. 7, panel C. When the restriction endonuclease cleaves the hybridized duplex, the right-hand portion of the target is only bound by a few hydrogen bonds and disassociates. Strand exchange now occurs in reverse. This portion of the lower polynucleotide generates a primer for the displacement polymerase.

Polymerase extension is shown in FIG. 7, panel D. A polymerase with strand displacement activity generates an exact copy of the target sequence and a target sequence for the RMD probe.

Polymerase extension continues, and rolling circle production of multiple copies of target and RMD target will ensue (FIG. 7, panel E). Multiple copies of RMD target sequence are generated, and can be detected as described herein.

### Example 7

### Synthesis of a Circular RC-PNCR Probe

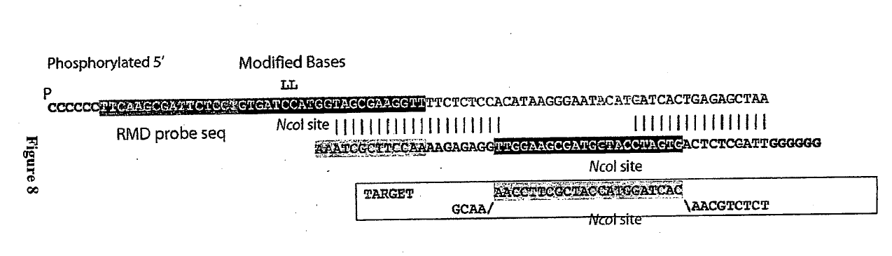

FIG. 8 shows an exemplary design for a probe for use in RC-PNCR. Self-ligation of multiple copies of this duplex molecule will generate a circular configuration. Modified nucleotides (typically Locked Nucleic Acids—LNAs) are included into the restriction endonuclease site on the circular polynucleotide to reduce cleavage by the NcoI enzyme. The probe consists of two oligonucleotides, oligonucleotide 1 (5′-CCCCCCTTCAAGCGATTCTCCTGTGATCCATGGTAG CGAAGGTTTTCTCTCCACATAAGGGAATACATGATCACTGAGAGCTAA-3′ [SEQ ID. NO. 3]), a region of which is complementary to a region of oligonucleotide 2 (5′-GGGGGGTTAGCTCTCAGTGATCCATGGTAGCGAAGGTT GGAGAGAAAACCTTCGCTAAA-3′[SEQ ID. NO. 4]).

This probe has been designed to detect a target sequence (5′-GCAAAACCTTCGCTACCATGGATCACAACGTCTCT-3′ [SEQ ID. NO. 5]) within the coliform genes encoding 3-isopropylmalate dehydrogenase.

It will be appreciated that the above description is provided by way of example only and that variations in both the materials and the techniques used which are known to those persons skilled in the art are contemplated.

### PUBLICATIONS

Mullis. 1987, U.S. Pat. No. 4,683,202. PROCESS FOR AMPLIFYING NUCLEIC ACID SEQUENCES.
Livak et al. 1998, U.S. Pat. No. 5,723,591. SELF-QUENCHING FLUORESCENCE PROBE.
Gelfand et al. 1993 U.S. Pat. No. 5,210,015. HOMOGENOUS ASSAY SYSTEM USING THE NUCLEASE ACTIVITY OF A NUCLEIC ACID POLYMERSASE.
Barany et al. 2006, U.S. Pat. No. 7,014,994. COUPLED POLYMERASE CHAIN REACTION-RESTRICTION-ENDONUCLEASE DIGESTION-LIGASE DETECTION REACTION PROCESS
Fisher et al. 1996, U.S. Pat. No. 5,491,063. METHODS FOR IN-SOLUTION QUENCHING OF FLUORESCENTLY-LABELLED OLIGONUCLEOTIDE PROBES
Kidwell. (1994), U.S. Pat. No. 5,332,659. LIGHT EMISSION OR ABSORBANCE-BASED BINDING ASSAYS FOR POLYNUCLEIC ACIDS
Matsumoto et al., U.S. Pat. No. 6,830,889. METHOD OF DETECTING DNA BY DNA HYBRIDIZATION METHOD WITH THE USE OF FLUORESCENT RESONANCE ENERGY TRANSFER
Coull et. Al. 2005, U.S. Pat. No. 6,949,343: METHODS, KITS AND COMPOSITIONS PERTAINING TO PNA MOLECULAR BEACONS
Barany et. Al. 1996, U.S. Pat. No. 5,494,810: THERMOSTABLE LIGASE-MEDIATED DNA AMPLIFICATIONS SYSTEM FOR THE DETECTION OF GENETIC DISEASE
Tyagi S. and Kramer F. (1996) MOLECULAR BEACONS: PROBES THAT FLUORESCE UPON HYBRIDIZATION. Nat. Biotechnology. 14: 303-308.
Barany F. (1991) THE LIGASE CHAIN REACTION IN A PCR WORLD. PCR Methods and Applications, vol. 1 pp. 5-16.
Broude N. (2005) MOLECULAR BEACONS AND OTHER HAIRPIN PROBES. Encyclopedia of Dignostic Genomics and Proteomics. Marcel Dekker Inc.
Peffer N., Hanvey J., Bisi J., Thomson S., Hassman, F., Noble, S, and Babiss, L. (1993) STRAND INVASION OF DUPLEX DNA BY PEPTIDE NUCLEIC ACID OLIGOMERS. Proc. Natl. Acad. Sci. USA. 90:10648-10652
Slaitas A., Ander C., Földes-Papp Z, Rigler R. and Yeheskiely E. (2003) SUPRESSION OF EXONUCLEOLYTIC DEGRADATION OF DOUBLE-STRANDED DNA AND INHIBITION OF EXONUCLEASE III by PNA. Nucleosides, Nucleotides and Nucleic Acids 22:1603-1605.

1. A method for detecting a target nucleic acid in a sample, the method comprising the steps
a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridisable to the first nucleic acid molecule, the second nucleic acid molecule comprising at least one copy of the target nucleic acid sequence, or a detection sequence, or both,
c) contacting the sample with more than one copy of the probe, wherein the target binding domain binds the target nucleic acid sequence,
d) contacting the sample with a first nuclease to cleave the nuclease cleavage element or degrade the target binding domain,
e) separating the first and second nucleic acid molecules of the dimeric probe to expose the at least one copy of the target nucleic acid sequence on the second nucleic acid molecule, or the detection sequence, or both, wherein this separation allows the target binding domain of the probe to bind the exposed at least one copy of the target nucleic acid sequence on the second nucleic acid molecule, and the exposure of additional copies of the at least one copy of the target nucleic acid sequence, or the detection sequence, or both, and
f) detecting the amount of the target nucleic acid sequence, or the detection sequence, or both.

2. A method according to claim 1 wherein the nuclease cleavage element comprises one strand of a restriction endonuclease recognition site, and the first nuclease is a restriction endonuclease.

3. A method according to claim 1 wherein the nuclease cleavage element comprises RNA and the first nuclease is an RNAase.

4. A method according to claim 3 wherein the first nuclease is RNAse H.

5. A method according to claim 1, wherein the separation of the first and second nucleic acid molecules of the dimeric probe is by exonucleolytic degradation of the first nucleic acid molecule by a second nuclease.

6. A method according to claim 1, wherein the separation of the first and second nucleic acid molecules of the dimeric probe is by strand displacement by a polymerase having strand displacement activity.

7. A method according to claim 1, wherein the first nucleic acid molecule contains a detectable label.

8. A method according to claim 7 wherein the signal of the detectable label is diminished or rendered undetectable when in sufficiently close proximity to a masking group, and the second nucleic acid molecule contains a masking group capable of diminishing or rendering undetectable the signal of the label when the dimeric probe is intact or when the first nucleic acid molecule is bound to the second nucleic acid molecule.

9. A method according to claim 1, wherein the method comprises the additional steps of
contacting the sample with a second probe that binds the detection sequence, the second probe carrying a detectable label, and
detecting or measuring the signal of the detectable label,
wherein an increase in signal is indicative of the presence of the target nucleic acid in the sample.

10. A method according to claim 9 wherein the second probe is a single stranded RNA probe comprising a fluorophore, a quencher and a detection sequence binding domain.

11.-31. (canceled)

32. A method for detecting a target nucleic acid in a sample, the method comprising the steps
a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the first nucleic acid molecule carrying a quencher and comprising at least one copy of the target nucleic acid sequence, the probe comprising a second nucleic acid molecule hybridisable to the first nucleic acid molecule, the second nucleic acid molecule carrying a fluorophore,
c) contacting the sample with an excess of the probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a fluorescent signal and exposing the at least one copy of the target nucleic acid sequence on the first nucleic acid molecule, wherein this exposing allows the target binding domain of the probe to bind the exposed at least one copy of the target nucleic acid sequence on the first nucleic acid molecule, and the amplification of the fluorescent signal and exposure of additional copies of the at least one copy of the target nucleic acid sequence, and
f) detecting or measuring the fluorescent signal,
wherein an increase in signal is indicative of the presence of the target nucleic acid in the sample.

33. (canceled)

34. A method for detecting a target nucleic acid in a sample, the method comprising the steps
a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second, circular nucleic acid molecule hybridisable to the first nucleic acid molecule, the second nucleic acid molecule comprising at least one copy of a sequence that is the reverse complement of the target nucleic acid sequence and at least one copy of a sequence that is the reverse complement of a detection sequence,
c) contacting the sample with more than one copy of the dimeric probe so the target binding domain binds the target nucleic acid sequence,
d) contacting the sample with a nuclease to cleave the nuclease cleavage element or degrade the target binding domain,
e) contacting the sample with a polymerase that binds the second nucleic acid molecule and displaces the first nucleic acid molecule from the second nucleic acid molecule, thereby generating a reverse complement of the second nucleic acid molecule, the reverse complement containing at least one copy of the target nucleic acid sequence and at least one copy of the detection sequence, wherein the generation allows the target binding domain of the probe to bind the exposed at least one copy of the target nucleic acid sequence and the exposure of additional copies of the at least one copy of the target nucleic acid sequence, or the detection sequence, or both,
f) contacting the sample with a second probe that binds the detection sequence, the second probe carrying a detectable label, and
g) detecting or measuring the signal of the detectable label, wherein an increase in signal is indicative of the presence of the target nucleic acid in the sample.

35. A method according to claim 34 wherein the nuclease cleavage element comprises one strand of a restriction endonuclease recognition site, and the first nuclease is a restriction endonuclease.

36. A method according to claim 34 wherein the nuclease cleavage element comprises RNA and the first nuclease is an RNAase.

37. A method according to claim 36 wherein the first nuclease is RNAse H.

38. A method according to claim 34 wherein the signal of the detectable label is diminished or rendered undetectable by a masking group when the second probe is not bound to the detection sequence.

39. A method according to claim 38 wherein the second probe is a single stranded RNA probe, the detectable label is a fluorophore, and the masking group is a quencher.

40. A method according to claim 39 wherein the method comprises the additional step of contacting the sample with an agent having RNAse H activity.

41.-45. (canceled)

46. A method for detecting a target nucleic acid in a sample, the method comprising the steps
a) providing a sample containing a target nucleic acid sequence,
b) providing a dimeric polynucleotide probe comprising a first nucleic acid molecule having a single stranded region, the single stranded region comprising a target binding domain, the target binding domain comprising a nuclease cleavage element or being susceptible to nuclease degradation, the probe comprising a second nucleic acid molecule hybridisable to the first nucleic acid molecule, the second nucleic acid molecule comprising at least one copy of a sequence that is the reverse complement of a detection sequence,
c) contacting the sample with more than one copy of the nucleic acid molecules comprising the dimeric probe so the target binding domain hinds the target nucleic acid sequence,
d) contacting the sample with a nuclease to cleave the nuclease cleavage element or degrade the target binding domain,
e) contacting the sample with a polymerase that binds the cleaved or degraded remainder of a first nucleic acid molecule bound to a second nucleic acid molecule, thereby synthesizing a reverse complement of the second nucleic acid molecule, the reverse complement containing at least one copy of the detection sequence,
f) contacting the sample with a second probe that binds the detection sequence, the second probe carrying a detectable label, and
g) detecting or measuring the signal of the detectable label, wherein an increase in signal is indicative of the presence of the target nucleic acid in the sample.

47. A method according to claim 46, wherein the first nucleic acid molecule of the dimeric probe is provided hybridized to the second nucleic acid molecule of the dimeric probe.

48. A method according to claim 46, wherein the first nucleic acid molecule and the second nucleic acid molecule are provided separately.

49. A method according to claim 46, wherein the second nucleic acid molecule is a circular nucleic acid molecule comprising at least one copy of a sequence that is the reverse complement of the target nucleic acid sequence and at least one copy of a sequence that is the reverse complement of a detection sequence.

50. A method according to claim 46, wherein the second nucleic acid molecule is a linear nucleic acid molecule comprising at least one copy of a sequence that is the reverse complement of the target nucleic acid sequence.

51. A method according to claim 46, wherein the method comprises the additional step of contacting the sample with a second nuclease to cleave or degrade the second nucleic acid molecule bound to its reverse complement.

52.-56. (canceled)
